(19) United States
(12) Patent Application Publication
(10) Pub. No.: US 2018/0250312 A1
(43) Pub. Date: Sep. 6, 2018

(54) TOPICAL CORTICOSTEROID COMPOSITIONS

(71) Applicant: PROMIUS PHARMA LLC, Princeton, NJ (US)

(72) Inventors: Sateesh KANDAVILLI, Plainsboro, NJ (US); Priyadarshani SAHUKAR, Telangana (IN); Franklin OKUMU, Morristown, NJ (US)

(21) Appl. No.: 15/958,368

(22) Filed: Apr. 20, 2018

Related U.S. Application Data
(63) Continuation of application No. 14/849,465, filed on Sep. 9, 2015.
Application No. 14/849,465 is a continuation-in-part of application No. 14/645,338, filed on Mar. 11, 2015.
(60) Provisional application No. 61/951,165, filed on Mar. 11, 2014.

(51) Int. Cl. A61K 31/573; A61K 31/57; A61K 47/20; A61K 9/10; A61K 9/00; A61K 47/10; A61K 9/12

(52) U.S. Cl. CPC A61K 31/573; A61K 31/57; A61K 47/20; A61K 9/10; A61K 47/10; A61K 9/12; A61K 9/0014

(57) ABSTRACT

Compositions for the topical administration of an active agent comprise a corticosteroid and a fatty alcohol as a skin penetration enhancer, in the form of topical sprays. Processes for preparing such compositions and methods of using them in management of skin diseases or disorders such as psoriasis, dermatoses, and other associated skin diseases or disorders, are described.

### CROSS-REFERENCE TO RELATED APPLICATIONS

The present application is a continuation of U.S. patent application Ser. No. 14/849,465, filed Sep. 9, 2015, which is a continuation-in-part of U.S. patent application Ser. No. 14/645,338, filed Mar. 11, 2015, which claims the benefit of U.S. Provisional Patent Application No. 61/951,165, filed Mar. 11, 2014, the disclosures of which are incorporated herein by reference.

### FIELD OF THE INVENTION

The present application relates to an aqueous based topical corticosteroid composition.

### BACKGROUND

Topical drug delivery systems are an ideal choice for treating various skin disorders locally. Topical dosage forms such as ointments, creams, gels, sprays, etc. are available to deliver the active agents to diseased area of the skin.

Inflammatory skin disorders are common in people of all age groups, races and genders, and these disorders are characterized by inflammation and irritation of the skin. Diagnosis and treatment of inflammatory skin disorders remains challenging in dermatological practice. Psoriasis is one of the inflammatory skin disorders. It is a chronic papulosquamous cutaneous disease which manifests through the appearance of red scaly patches on the skin. It generally affects the elbows, knees, and scalp. Although many therapeutic choices are available to treat psoriasis such as: topical therapy, phototherapy and systemic therapy, topical corticosteroids are the first choice for treating psoriasis. Phototherapy and systemic therapy are secondary and they are generally preferred when topical corticosteroids fail in treating psoriasis.

Corticosteroids are widely used in clinical practice. In particular, topical corticosteroids have been used to treat various skin conditions such as psoriasis, dermatitis, etc. Corticosteroids are chemically classified into hydrocortisone type, acetonide type, betamethasone type, etc. They are also classified based on their potency in the Vasoconstrictor assay (VCA), otherwise called the skin blanching assay.

The VCA is often used to access the potency of topically administered corticosteroids and to determine the bioequivalence of topically administered corticosteroids as U.S. Food and Drug Administration (FDA) guidance for industry. Accordingly, corticosteroids can be classified by VCA as super potent (Class 1), high potent (Class 2), upper mid strength (Class 3), mid strength (Class 4), lower mid strength (Class 5), low potent (Class 6), and least potent (Class 7).

The drug compound having the adopted name “betamethasone dipropionate” belongs to super potent (Class 1) and/or high potent (Class 2) and has a chemical name 9-fluoro-11(β),17,21-trihydroxy-16(β)-methylpregna-1,4-diene-3,20-dione 17,21-dipropionate and is represented by structural Formula I.

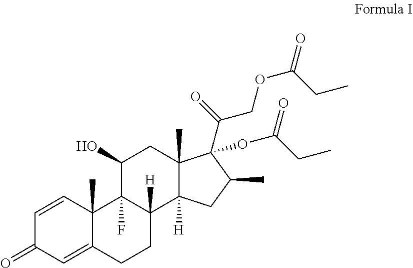

Betamethasone dipropionate is a white to cream-white powder. It is practically insoluble in water, sparingly soluble in ethanol and freely soluble in acetone and chloroform.

Topical betamethasone dosage forms, such as aerosol foam, cream, ointment, gel, and lotion formulations are commercially available. Combination formulations of betamethasone dipropionate with calcipotriene hydrate and also with clotrimazole exist. Betamethasone dipropionate is the active ingredient in commercially available products sold as DIPROLENE AF® and DIPROLENE® that comprise 0.05% betamethasone compound, and are intended for application to affected skin areas once or twice daily.

Some topical corticosteroids are administered as occlusive dosage forms, which cause stratum corneum to hydrate thereby improving penetration of corticosteroidal drug into skin layers. The ointment dosage form has greater absorption because of the occlusive nature of the ointment base, however, which creates greasy sensation to subjects. Moreover, it is necessary to rub such formulations into the target site to improve the penetration of the active agent into the epidermis, an action which itself produces irritation. In addition, the presence of alcohol causes irritation/stinging to subject skin, and solution based topical compositions have tendency to evaporate before the active agent penetrates the epidermis. Propellant-containing topical aerosol compositions, in the market, are priced relatively higher than their counterparts.

The stratum corneum (SC) is the first layer of the skin comprising dead cells and provides the rate limiting step in percutaneous absorption of drugs through the skin layers. There are only a few drugs that possess the physiochemical properties necessary to penetrate the stratum corneum. Percutaneous absorption involves the passage of the drug molecule from the skin surface into the stratum corneum under the influence of a concentration gradient and the drug molecule's subsequent diffusion through the stratum corneum and underlying epidermis, through the dermis.

The nature of the vehicle in a topical composition has a profound effect on the rate of release and delivery of the agent in the skin layers passage through the stratum corneum. The vehicle used to deliver the drug can aid in the efficacy and stability of the product. Generally aqueous vehicles are preferred in topical dosage form due to their non-irritant, superior tolerability and non-toxic nature. An important aspect here is that most of corticosteroid drugs are exceptionally poorly soluble in aqueous vehicles and cause instability.

Another important aspect in a topical composition is the inclusion of a substance which assists the active agent to penetrate or diffuse through the skin layers, i.e., “skin penetration enhancers.” Skin penetration enhancers are necessary for the active to penetrate in the skin layers. Various classes of skin penetration enhancers are available, such as, fatty acids and their esters, pyrrolidones, sulfoxides, glycols, glycerides, etc. However, skin penetration enhancers are known to act differently with different active agent.

Conventionally, topical coricosteroid products are available as creams, lotions and ointments. U.S. Pat. No. 3,892,856 describes the use of corticosteroids dissolved in polyethylene glycol and emulsified into an oleaginous base.

U.S. Pat. No. 3,934,013 describes topical pharmaceutical compositions containing at least two corticosteroids, propylene glycol, a fatty alcohol and water. The patentee describes the “fatty alcohol ingredient” as any fatty alcohol having from 16-24 carbon atoms and, preferably, as a saturated, monohydric primary alcohol such as cetyl alcohol, stearyl alcohol or behenyl alcohol.

U.S. Pat. No. 4,343,798 discloses topical antimicrobial/anti-inflammatory compositions containing C₅-C₁₂ fatty acids in combination with corticosteroids.

PCT application WO 2011/026076 discloses pharmaceutically topical sprayable compositions comprising steroid as active agent.

U.S. Pat. Nos. 6,126,920 and 7,078,058 disclose betamethasone valerate aerosols with a quick-break foaming agent, a propellant, and a buffering agent, wherein ethanol is present.

U.S. Pat. No. 5,369,131 discloses a liquid mechanically foamable pharmaceutical composition, which is propellant free, for local application.

U.S. Patent Application Publication No. 2008/0102039 discloses spray foaming dosage compositions comprising propylene glycol.

U.S. Pat. No. 5,958,379 discloses a pharmaceutical composition that is sprayable as liquid droplets, forming a preparation within times less than 4 seconds.

U.S. Patent Application Publication No. 2006/0239929 discloses a sprayable composition for the treatment of psoriasis, comprising clobetasol as the active agent together with ethyl alcohol.

There is an unmet need for subject compliant topical formulations that are effective in the treatment of skin disorders such as psoriasis, and which provide improved delivery of the active agent at the desired site of action, with decreased inconvenience and irritation, increased ease of use for the subject, and longer duration of action. Topical spray compositions are always preferred over any other topical dosage forms due to subject acceptance and convenience of application in skin area.

Topical corticosteroids are widely approved for use in various skin disorders and topical corticosteroids are known to have solubility issues such that corticosteroids are insoluble in water or aqueous solvents. The propylene glycol is an essential solvent and/or cosolvent in the topical compositions containing corticosteroids. It is widely known for solubilizing corticosteroids and acts as cosolvent to facilitate solubility of corticosteroids in the topical compositions. Furthermore propylene glycol is known as better skin penetration enhancer for corticosteroids. Propylene glycol is used in more than 100 approved topical compositions comprising corticosteroids. On the other hand propylene glycol causes significant allergy and skin irritation to the subject's skin.

Aqueous based topical spray composition of the present application is formulated to achieve equal or superior efficacy to marketed products and to overcome the shortcomings of subject compliance in the use of topical dosage forms.

### SUMMARY OF THE INVENTION

An aspect of the present application relates to an aqueous based topical spray composition comprising: a) a corticosteroid; b) at least one fatty alcohol; c) at least one pharmaceutically and/or dermatologically acceptable excipient; and d) water.

Another aspect of the present application relates to use of an aqueous based topical spray compositions comprising a corticosteroid as an active agent for prophylaxis, amelioration, or treatment of psoriasis, corticosteroid responsive dermatoses, erythema, contact sensitivity reactions, and other associated diseases or disorders.

Another aspect of the present application relates a method of treating skin diseases, which comprises administering an aqueous-based topical spray composition comprising a betamethasone compound once or twice daily to an affected area of skin of a subject, wherein the composition is administered for at least 1, 2, 3, 4, 5, 6, 7, 8, 9, 10, 11, 12, 13, 14, 15, 16, 17, 18, 19, 20, 21, 22, 23, 24, 25, 26, 27, 28 or up to 29 days which provides hypothalamic-pituitary-adrenal (HPA) axis suppression substantially lower or on part with that of Diprolene lotion, 0.05%.

Another aspect of the present application relates a method of treating skin diseases, which comprises administering an aqueous-based topical spray composition comprising a betamethasone compound once or twice daily to an affected area of skin of a subject, wherein the composition is administered for at least 1, 2, 3, 4, 5, 6, 7, 8, 9, 10, 11, 12, 13, 14, 15, 16, 17, 18, 19, 20, 21, 22, 23, 24, 25, 26, 27, 28 or up to 29 days.

In another aspect, an aqueous based topical spray composition of present application can be administered for more than 1, 2, 3, 4, 5, 6, 7, 8, 9, 10, 11, 12, 13, 14, 15, 16, 17, 18, 19, 20, 21, 22, 23, 24, 25, 26, 27, 28 or up to 29 days based on severity of the disease condition with substantially no hypothalamic-pituitary-adrenal (HPA) axis suppression.

One embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, once or twice daily to the affected area of skin of the subject, wherein the composition is administered at least for a day, for up to 15 days, or for up to 29 days, which provides HPA-axis suppression lower or at par with Diprolene lotion, 0.05%.

Another embodiment of the present application relates to a method of treating skin diseases or disorders such as psoriasis, steroid responsive dermatoses, erythema, contact sensitivity reactions, and other associated diseases or disorders comprising administration of topical spray composition comprising betamethasone compound, once or twice daily to the affected area of skin of the subject, wherein the composition can be administered up to 29 days based on severity of the disease condition with substantially no HPA-axis suppression.

In another embodiment, the topical spray composition of the present application comprising betamethasone compound provides ‘mean Cmax’ of betamethasone-17, 21-dipropionate, betamethasone-17-propionate and betamethasone base (sum of ‘mean Cmax’ values of individual products) of less than about 400 pg/ml, when administered twice daily to a subject for 15 days, or for 29 days.

In another aspect ‘mean Cmax’ is in the range of about 5 pg/ml to about 30 pg/ml, about about 5 pg/ml to about 50 pg/ml, 5 pg/ml to about 75 pg/ml, about 5 pg/ml to about 100 pg/ml, about 10 pg/ml to about 300 pg/ml, about 10 pg/ml to about 150 pg/ml, about 10 pg/ml to about 275 pg/ml, about 20 pg/ml to about 90 pg/ml, about 30 pg/ml to about 125 pg/ml, about 50 pg/ml to about 290 pg/ml, about 20 pg/ml to about 250 pg/ml, about 50 pg/ml to about 200 pg/ml

In one aspect of the above embodiment, ‘mean Cmax’ is less than about 100 pg/ml, less than about 150 pg/ml, less than about 250 pg/ml, less than about 300 pg/ml.

In another aspect, ‘mean Cmax’ is not measurable (<5 pg/ml).

In another aspect, an aqueous based topical spray composition of present application comprises: a) a betamethasone compound; b) oleyl alcohol; c) at least one pharmaceutically and/or dermatologically acceptable excipient; and d) water.

Another aspect of the present application relates to a process for preparing an aqueous based topical spray composition, comprising: a) heating a mixture comprising an emulsifying agent and a water-immiscible substance to obtain an oily phase; b) optionally, mixing an antioxidant, preservative, or both with the oily phase of a); c) mixing an active agent with a penetration enhancer; d) mixing the material of c) with the mixture of a) or b); e) dissolving a polymer in water to form an aqueous phase; and f) mixing the oily phase of d) with an aqueous phase of e), to form an emulsion.

### BRIEF DESCRIPTION OF THE DRAWINGS

FIG. 1 shows the structures of certain betamethasone propionate impurities.

FIG. 2 shows mean irritation score of exemplary Composition 6 in comparison with other vehicles in Irritation Patch Test Study of betamethasone dipropionate spray.

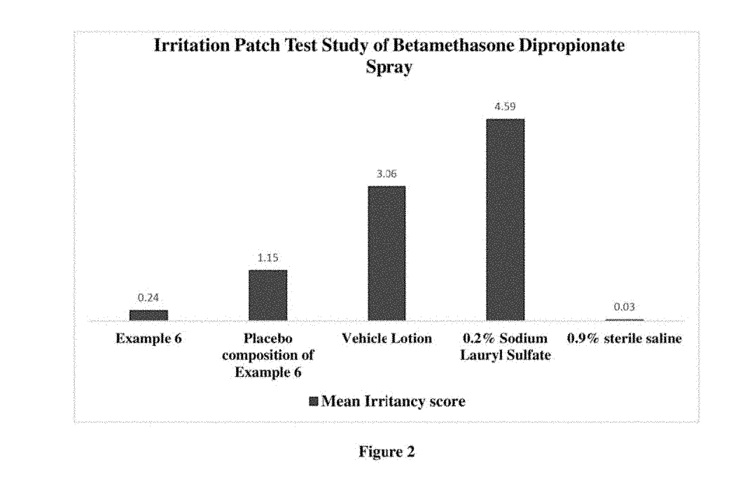

FIG. 3 shows amounts of betamethasones retained in individual skin layers by exemplary Compositions 1-6.

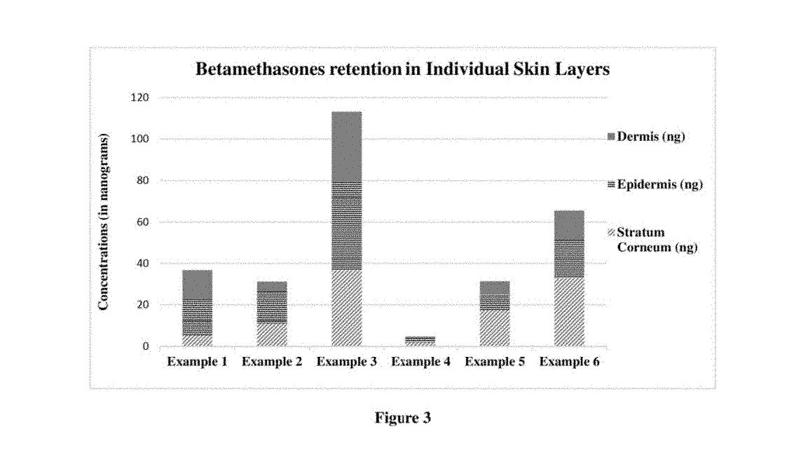

FIG. 4 shows percentage of betamethasones retained in skin layers in comparison with receptor level by exemplary Compositions 1-6.

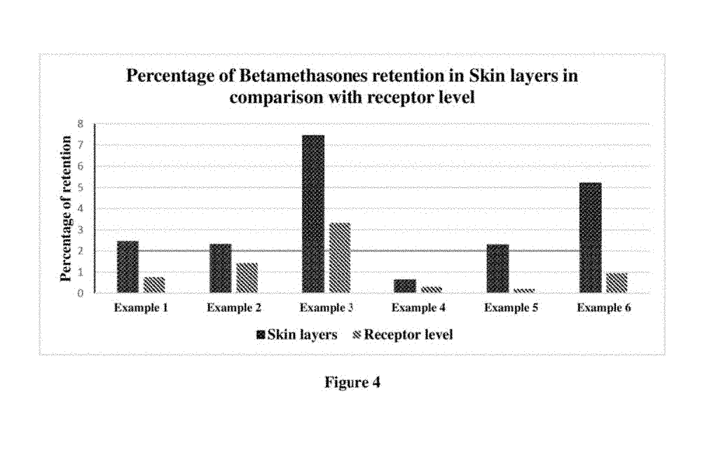

FIG. 5 shows amounts of betamethasones permeated through receptor level by exemplary Compositions 1-6.

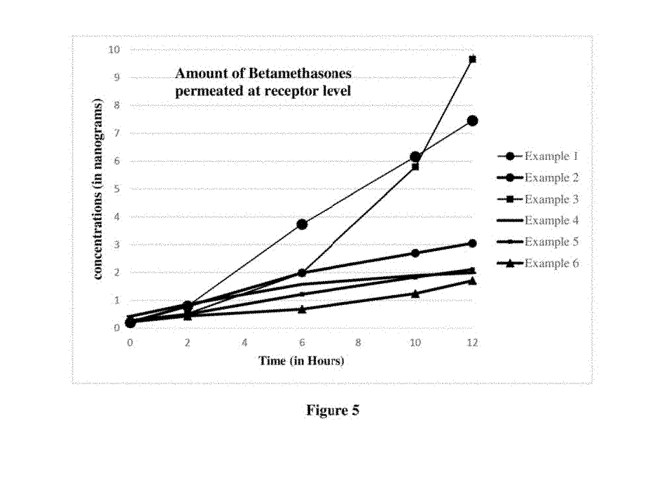

### DETAILED DESCRIPTION OF THE INVENTION

The term “stable” as used herein refers to physical stability and/or chemical stability of the active agent in a topical composition, wherein changes in the drug assay values and/or impurities content are less than about 10%, during stability study storage of the composition at 25° C. and 60% relative humidity (RH), or 30° C. and 65% RH, or 40° C. and 75% RH, for durations such as 3, 6, 12, 18 or 24 months.

The term “propellant free” or “free of propellant(s)” as used herein indicates that the compositions are not delivered using any of the commonly used aerosol propellants, such as fluorochloro hydrocarbons, hydrocarbons, compressed gases, and the like.

As used herein, “Cmax” refers to maximum plasma concentration of an individual subject. As used herein, “mean Cmax” refers to mean maximum plasma concentration, and “median Cmax” refers to median maximum plasma

The term “substantially free” as used herein indicates that the specified substance referred to is present in amounts not more than 10% by weight of the total composition or in amounts not more than about 9% by weight of the total composition, or in amounts not more than about 8% by weight of the total composition, or in amounts not more than about 7% by weight of the total composition, or in amounts not more than about 6% by weight of the total composition, or in amounts not more than about 5% by weight of the total composition, or in amounts not more than about 4% by weight of the total composition, or in amounts not more than about 3% by weight of the total composition, or in amounts not more than about 2% by weight of the total composition or in amounts not more than about 1% by weight of the total composition or in an amount about 0% by weight of the total composition or completely free of specified substance (i.e.,) 0%.

The term “substantially non-foaming” as used herein indicates that the topical spray composition forms mist or droplet in more than 90% of quantity, when sprayed on to the affected skin area. Preferably, the topical spray composition forms mist or droplet in more than 95% of quantity, when sprayed on to the affected skin area.

The term “substantially non-irritating” as used herein indicates that an aqueous based topical spray composition of the present application does not cause erythema, papules, definite edema, vesicular eruption at test site, and any noticeable strong reaction which is spreading beyond test site even in semi occlusive conditions.

The term “substantially no hypothalamic-pituitary-adrenal (HPA) axis suppression” refers to a relative retention of HPA axis activity, but does not require an absolute retention of 100% activity. In some embodiments, “substantially no HPA axis suppression” refers to an activity suppression of less than about 0.1, 0.5, 1, 2, 3, 4, 5, 6, 7, 8, 9, or 10%. In some embodiments, the term refers to suppression of 0%, i.e., a retention of 100% activity.

The term “about” as used herein when referring to a specified value or amount is meant to encompass variations of, in some embodiments ±10%, in some embodiments ±5%, in some embodiments ±1%, in some embodiments ±0.5%, and in some embodiments ±0.1% from the specified value or amount.

A “skin permeation enhancer” or “skin penetration enhancer” or “penetration enhancer” is a component used to enhance the penetration rate of drugs through the skin or mucous membrane, such as by temporarily diminishing the impermeability of the skin or membrane. Permeation enhancers have also been called “accelerants” and “absorption promoters.” There are numerous penetration enhancers that can be used. It has been found that when fatty alcohols reduce permeation lag time thereby enhancing the delivery into epidermis and dermis. In an aspect of the present application, the skin penetration enhancer, without any limitation, is selected from C₅-C₄₄ fatty alcohols, preferably C₅-C₂₀ fatty alcohols. These fatty alcohols belong to the group of long chain saturated fatty alcohols, unsaturated chain fatty alcohol, branched chain alcohol or combinations thereof.

“Emollients” are substances that soften and soothe the skin. They are used to correct dryness and scaling of the skin. Various emollients include, but are not limited to, oils of natural origin such as almond oil, coconut oil, olive oil, palm oil, peanut oil and the like; fatty acids such as lauric acid, myristic acid, palmitic acid, and stearic acid; monohydric alcohol esters of the fatty acids such as ethyl laurate, isopropyl laurate, ethyl myristate, n-propyl myristate, isopropyl myristate, ethyl palmitate, isopropyl palmitate, methyl palmitate, methyl stearate, ethyl stearate, isopropyl stearate, butyl stearate, isobutyl stearate, amyl stearate, and isoamyl stearate; mineral oil and any combinations thereof.

The term “aqueous based” as used herein indicates that the carrier of the topical spray composition comprises a majority of water in the composition. In an aspect, the term “aqueous based” as used herein denotes that the topical spray composition comprising at least about 60% w/w or at least about 70% w/w of water based on total weight of the composition or at least about 75% w/w of water based on total weight of the composition.

In an aspect, the aqueous based topical spray composition comprising: a) a betamethasone compound; b) an oil phase comprising: i) at least fatty alcohol comprising oleyl alcohol and ii) an emulsifying agent; and c) an aqueous phase comprising: water; wherein the weight ratio between oil phase and aqueous phase is from about 1:1.5 to about 1:4; and the composition is skin depot composition.

In an aspect, the weight ratio between oil phase and aqueous phase is from about 1:1.5 to about 1:4. In a specific aspect, the weight ratio between oil phase to aqueous phase is selected from about 1:1.5 or about 1:1.6 or about 1:1.7 or about 1:1.8 or about 1:1.9 or about 1:2 or about 1:2.1 or about 1:2.2 or about 1:2.3 or about 1:2.4 or about 1:2.5 or about 1:2.6 or about 1:2.7 or about 1:2.8 or about 1:2.9 or about 1:3 or about 1:3.1 or about 1:3.2 or about 1:3.3 or about 1:3.4 or about 1:3.5 or about 1:3.6 or about 1:3.7 or about 1:3.8 or about 1:3.9 or about 1:4.

The term “carrier” denotes organic or inorganic ingredients, natural or synthetic, with which an active ingredient is combined to facilitate application of a composition. In the present context, the terms “carrier” and “vehicle” are interchangeably used. The term “carrier” includes, but is not limited to, water, acetone, alone or in combination with materials such as silicone fluids. The amounts of carrier are about 5% to about 99% of the total weight of the composition. In an aspect, a carrier according to the present application comprises water. In an aspect, the carrier can comprise, in addition to water, water-immiscible substances such as any pharmaceutically and/or dermatologically acceptable fatty esters of natural fatty acids, triglycerides of animal or vegetable, medium chain triglycerides, mixtures of mono-, di- and/or triglycerides, waxes, hydrogenated vegetable oils, and mixtures thereof.

The term “preservative” refers to a natural or synthetic chemical that is added to products to prevent decomposition by microbial growth or by undesirable chemical changes. Preservatives can desirably be incorporated into a composition for protecting against the growth of potentially harmful microorganisms. While microorganisms tend to grow in an aqueous phase, microorganisms can also reside in a hydrophobic or oil phase. Suitable preservatives for compositions of the present application include, but are not limited to, methylparaben, propylparaben, benzyl alcohol, chlorocresol, benzalkonium chloride, cetrimonium chloride, sodium edetate, boric acid, and any mixtures thereof. The amount of preservative is from about 0.25% to about 25% of the total weight of the composition.

The term “betamethasone compound” represents, but not limited to, betamethasone base, betamethasone dipropionate, betamethasone valerate, betamethasone acetate, betamethasone benzoate, betamethasone dipropionate, betamethasone sodium phosphate, betamethasone valerate, betamethasone sodium phosphate, betamethasone-17-propionate, betamethasone-21-propionate, and betamethasone-17-propionate 21-acetate and/or mixtures thereof. Unless otherwise specified, betamethasone compound intended to include its isomers, its metabolites, its salts, its esters, its derivatives or its prodrugs thereof.

The term “betamethasones” represents betamethasone dipropionate (betamethasone-17, 21-dipropionate), betamethasone-17-propionate, betamethasone-21-propionate, betamethasone base and/or mixtures thereof.

The term “corticosteroid” represents a compound selected from the group comprising of: alclometasone dipropionate, amcinonide, beclomethasone dipropionate, betamethasone, betamethasone benzoate, betamethasone dipropionate, betamethasone sodium phosphate, betamethasone valerate, betamethasone-17-monopropionatye, betamethasone-21-monopropionate, budesonide, clobetasol propionate, clobetasone butyrate, clocortolone pivalate, desonide, desoximetasone, dexamethasone, dexamethasone acetate, dexamethasone nicotinate, dexamethasone propionate, dexamethasone sodium phosphate, dexamethasone valerate, diflorasone diacetate, diflucortolone valerate, fluandrenolide, flumethasone pivalate, fluocinolone acetonide, fluocinonide, fluocortin butyl ester, fluticasone propionate, halcinonide, halobetasol propionate, halometasone monohydrate, hydrocortisone, hydrocortisone sodium phosphate, hydrocortisone sodium succinate, hydrocortisone-17-butyrate-21-propionate, hydrocortisone aceponate, hydrocortisone acetate, hydrcortisone valerate, hydrocortisone butyrate, hydrocortisone probutate, methylprednisolone, methylprednisolone acetate, methylprednisolone aceponate, mometasone furoate, prednisolone, prednisolone sodium phosphate, prednisolone acetate, prednisolone-17-valerate-21-acetate, triamcinolone acetonide, triamcinolone acetate, triamcinolone diacetate, and prednicarbate. Unless otherwise specified, recitation of corticosteroid or specific compound is intended to include the specific compound or any salts, esters, isomersmetabolites, conjugates, derivatives or prodrugs thereof. Betamethasone-17-propionate, betamethasone-21-propionate and betamethasone are the metabolites of the parent drug betamethasone dipropionate.

“Solvent” refers to components that aid in the dissolution of the drug in the composition. Solvents serve to maintain a solution of the drug in the composition. Some solvents can also enhance percutaneous penetration of drug and/or act as humectants. For corticosteroid drugs, solvents can include water-immiscible substances such as fatty esters of natural fatty acids, triglycerides of animal or vegetable, medium chain triglycerides, mixtures of mono-, di- and/or triglycerides, waxes, hydrogenated vegetable oils, and mixtures thereof. Some specific examples include castor oil, lanolin oil, citrate triisocetyl triglycerides having 10-18 carbon atoms, caprylic/capric triglycerides, coconut oil, corn oil, cottonseed oil, linseed oil, oil of mink, olive oil, palm oil, sunflower oil, nut oil, saturated paraffin oils, light or heavy mineral oils, vegetable oils, glycerides, and the like, and/or their mixtures thereof.

The term “applied dose” as used herein means the amount of topical spray composition dispensed to the subject skin in one actuation of topical spray device. For example, if an applied dose is about 170 mg which contains about 0.064% w/w of betamethasone dipropionate (about 0.108 mg), the percentage retention of betamethasones in skin layer is about 0.1% to about 10% of 0.108 mg of betamethasone dipropionate.

The term “skin depot” as used herein refers to a topical composition which provides higher skin retention of the applied drug compared to systemic exposure of the same drug.

The term “skin layers” as used herein includes stratum corneum, epidermis and dermis of mammalian skin.

The term “systemic exposure” as used herein includes tendency of any topically applied drugs entering into systemic circulation of mammals, thereby causing systemic side effects, for example, as will be recognized by one of ordinary skill in the art, corticosteroids cause systemic side effects of hypothalamic-pituitary-adrenal (HPA) axis suppression.

The term “emulsifying agent”, “surfactant” and “emulsifier” are used interchangeably.

Various aspects of the present application relate to an aqueous based topical spray composition comprising a corticosteroid.

Further aspects of the present application relate to an aqueous based topical spray composition comprising a Betamethasone compound.

Still further aspects of the present application relate to an aqueous based topical spray composition comprising a Betamethasone dipropionate.

An aspect of the present invention relates to an aqueous based topical spray composition comprises: a) a corticosteroid; b) at least one fatty alcohol; c) at least one pharmaceutically and/or dermatologically acceptable excipient(s); and d) water.

An aspect of the present invention relates to an aqueous based topical spray composition comprises: a) a betamethasone compound; b) at least one fatty alcohol; c) at least one pharmaceutically and/or dermatologically acceptable excipient(s); and d) water.

In one aspect, the fatty alcohol in the above composition is acting as a skin penetration enhancer or a skin permeation enhancer.

In another aspect, the fatty alcohol is C₅-C₂₀ fatty alcohols.

In another aspect, these fatty alcohols selected from saturated fatty alcohols, unsaturated fatty alcohols, branched chain fatty alcohols and mixtures thereof.

In another aspect, the fatty alcohol is selected from the group comprising of, but not limited to, elaidyl alcohol, linoleyl alcohol, linolenyl alcohol, caproic alcohol, lauryl alcohol, stearyl alcohol, cetostearyl alcohol, behenyl alcohol, oleyl alcohol, 2-heptyl-1-undecanol, 1,17-hepatadecanediol, and combinations thereof.

In another aspect, the composition is oil-in-water emulsion.

In another aspect, the composition is substantially free of (C₁-C₄) alcohol.

In another aspect, the composition is free of propellants.

In another aspect, the composition of the present application is non-irritating to the skin, non-toxic and well-tolerated.

In another aspect, the corticosteroid is selected from the group comprising of betamethasone, clobetasol, halobetasol, clocortolone, desonide, triamcinolone, mometasone, alclometasone, and hydrocortisone. The corticosteroid may present as its acid or base form, its salt form, its ester form, its isomeric form, or in prodrug form.

In another aspect, the corticosteroid present in the composition of the present application is betamethasone compound, or a salt, an ester, an isomer, a derivative or a prodrug thereof.

In another aspect, the betamethasone compound is selected from the group comprising of betamethasone benzoate, betamethasone dipropionate, betamethasone sodium phosphate, betamethasone valerate, and combinations thereof.

In another aspect, the betamethasone compound is in the form of betamethasone dipropionate.

In another aspect, the corticosteroid present in the composition of the present application is mometasone furoate.

In another aspect, the corticosteroid present in the composition of the present application is betamethasone valerate.

In another aspect, the corticosteroid present in the composition of the present application is triamcinolone acetonide.

In another aspect, the corticosteroid present in the composition of the present application is alclometasone dipropionate.

An aspect of the present application relates to use of an aqueous based topical spray composition comprising a corticosteroid for prophylaxis, amelioration, or treatment of psoriasis, corticosteroid responsive dermatoses, erythema, contact sensitivity reactions, and other associated diseases or disorders.

An aspect of the present invention related to use of an aqueous based topical spray composition comprising a corticosteroid for prophylaxis, amelioration, or treatment of moderate to severe plaque psoriasis.

An aspect of the present invention related to use of an aqueous based topical spray composition comprising a corticosteroid for prophylaxis, amelioration or treatment of moderate plaque psoriasis.

An aspect of the present application relates to a topical spray composition comprising: a) a betamethasone compound; b) at least one fatty alcohol comprising: oleyl alcohol in the range of from about 0.1% w/w to about 10% w/w; c) a polymer in the range of from about 0.01% w/w to about 1% w/w; d) an emulsifying agent; e) water and f) at least one pharmaceutically acceptable excipient; wherein the composition is aqueous based emulsion; substantially free of propellant, glycols and alcohol; the composition is non-foaming and the composition having pourable liquid like consistency and viscosity of from about 100 cps to about 1000 cps.

An aspect of the present application relates to use of an aqueous based topical spray composition comprising a betamethasone compound for prophylaxis, amelioration, or treatment of psoriasis, corticosteroid responsive dermatoses, erythema, contact sensitivity reactions, and other associated diseases or disorders.

In another aspect, the betamethasone compound is betamethasone dipropionate.

An aspect of the present application related to use of an aqueous based topical spray composition comprising a betamethasone compound for prophylaxis, amelioration, or treatment of moderate to severe plaque psoriasis.

In another aspect, the betamethasone compound is betamethasone dipropionate.

An aspect of the present application related to use of an aqueous based topical spray composition comprising a betamethasone compound for prophylaxis, amelioration or treatment of moderate plaque psoriasis.

In another aspect, the betamethasone compound is betamethasone dipropionate.

In another aspect, the concentration of the betamethasone compound in the composition of the present application is from about 0.01% to about 10%, or from about 0.025% to about 5%, or from about 0.025% to about 0.5%, by weight based on the total weight of the composition.

A specific aspect of the application relates to an aqueous based topical spray composition comprising a betamethasone compound, in amounts equivalent to about 0.025 to about 0.1 percent by weight of the product.

Another aspect of the application relates to an aqueous based topical spray composition comprising a betamethasone compound, in amounts equivalent to about 0.05 by weight of the composition.

In another aspect, the betamethasone compound is betamethasone dipropionate.

In another aspect of the present application, weight of the betamethasone dipropionate is about 0.643 g.

In another aspect of the present application, 0.643 g of the betamethasone dipropionate is equivalent to 0.5 mg of betamethasone base.

In another aspect of the present application, fatty alcohol is present in an amount of about 0.001% to about 15% percent by weight based on the total weight of the composition.

An aspect of the present invention relates to an aqueous based topical spray composition comprising: a corticosteroid, a skin penetration enhancer, an emulsifying agent, a polymer, water, and a water-immiscible substance, wherein the skin penetration enhancer is present in the amount of about 0.001% to about 15% percent by weight based on the total weight of the composition.

In another aspect, the skin penetration enhancer is present in an amount of about 0.05% to about 12%, specifically about 3% to about 10% by weight based on the total weight of the composition.

In another aspect of the present application, an aqueous based topical spray composition comprising: a) betamethasone dipropionate; b) an oleyl alcohol; c) at least one pharmaceutically and/or dermatologically acceptable excipient; and d) water.

In a further aspect of the present application, an aqueous based topical spray composition comprising: a) betamethasone dipropionate; b) at least one fatty alcohol; c) at least one pharmaceutically and/or dermatologically acceptable excipient; and d) water, wherein said the fatty alcohol is selected from elaidyl alcohol, linoleyl alcohol, linolenyl alcohol, caproic alcohol, lauryl alcohol, stearyl alcohol, cetostearyl alcohol, behenyl alcohol, oleyl alcohol, 2-heptyl-1-undecanol, 1,17-hepatadecanediol and mixtures thereof, and wherein said topical spray composition is substantially free of (C₁-C₄) alcohol and free of propellants.

In another aspect, the composition further comprises emulsifying agent.

In another aspect, the composition does not form any film layer.

In another aspect, the composition is oil-in-water emulsion,

In another aspect, the above composition is substantially free of (C₁-C₄) alcohol.

In another aspect, the composition is free of propellants.

Another aspect of the present application relates to a process for preparing a topical spray composition of the present application, comprising: a) heating a mixture comprising an emulsifying agent and a water-immiscible substance to obtain an oily phase; b) optionally, mixing an antioxidant, preservative, or both with the oily phase of a); c) mixing an active agent with a penetration enhancer; d) mixing the material of c) with the mixture of a) or b); e) dissolving a polymer in water to form an aqueous phase; and f) mixing the oily phase of d) with an aqueous phase of e), to form an emulsion.

In further aspect, an aqueous based topical spray composition of the present application is substantially non-irritating to the skin, non-toxic and well-tolerated, thereby providing a high degree of subject compliance, and is useful in the prophylaxis, amelioration or treatment of skin diseases or disorders such as psoriasis, steroid responsive dermatoses, erythema, contact sensitivity reactions, and other associated diseases or disorders.

In another aspect, composition of the present application relates to sustained release of the corticosteroid, for better skin permeation and subject comfort.

In another aspect, composition of the present application relates to sustained release of the betamethasone compound, for better skin permeation and subject comfort.

In another aspect, composition of the present application relates to sustained release of the betamethasone dipropionate, for better skin permeation and subject comfort.

In an aspect, the present application provides method of using propellant-free topical spray composition comprising at least one corticosteroid as an active agent, wherein the method comprising administering a pharmaceutically and/or dermatologically effective amount of a spray composition directly onto an affected part of the skin of a subject in need thereof.

In another aspect, the present application provides method of using propellant-free topical spray composition comprising betamethasone compound, wherein the method comprising administering a pharmaceutically and/or dermatologically effective amount of a spray composition directly onto an affected part of the skin of a subject in need thereof.

In another aspect, the present application provides method of using propellant-free topical spray composition comprising betamethasone dipropionate, wherein the method comprising administering a pharmaceutically and/or dermatologically effective amount of a spray composition directly onto an affected part of the skin of a subject in need thereof.

In another aspect, topical spray composition of the present application is useful in the management of psoriasis, and further can provide a moisturizing and/or soothing effect at the site of application to the skin.

In another aspect, the composition reduces the dryness that accompanies the build-up of skin in psoriatic plaques.

In another aspect, the composition of the present application can be applied directly to the psoriatic lesions or dermatoses and can help reduce inflammation, remove built-up scale, reduce skin turnover, and/or clear affected skin of plaques.

Vasoconstriction assay (VCA) is used to measure dermatological potency of the topical corticosteroids and it is a recommended method to access in vivo bioequivalence of topical corticosteroid by US FDA (ref: Guidance for industry; Topical dermatological corticosteroids: in vivo Bioequivalence; Dated Jun. 2, 1995).

VCA study is performed in vivo and results are obtained based on blanching effect of the skin by two methods, one is chromameter method and the other one is visual scoring method. VCA is often considered for accessing potency, however, the result of the VCA study depends on the concentration of drug in stratum corneum and epidermis. [012$] The fatty alcohols contain at least one primary alcohol group in long chain hydrocarbons (C₅-C₄₄) and are derived from natural sources as well as synthetically made from fatty acids. Fatty alcohols are widely used in cosmetic and pharmaceutical industry in the preparation of topical drug compositions and cosmetic preparations such as hair care products, conditioners etc. Fatty alcohols are used as emollients, skin penetration enhancers, emulsifiers and thickeners. Unsaturated fatty alcohols contain, in addition to the primary alcohol group, at least one olefinic group in the chain and additionally they have “Z” (cis) and “E” (trans) configuration at the olefinic group in the chain. The physical and chemical properties of the fatty alcohols greatly vary depending on length of the chain and/or the presence or absence of the olefinic group in the chain. The selection and usability of the fatty alcohols depend mainly on the choice of active agent since fatty alcohols are known to act differently with different active agents due the chemical nature of the active agent. In another aspect, the fatty alcohols contain at least one primary alcohol group in long chain hydrocarbons (C₅-C₂₀).

In an aspect of the invention, the skin penetration enhancer is selected from the group comprising of elaidyl alcohol, linoleyl alcohol, linolenyl alcohol, caproic alcohol, lauryl alcohol, stearyl alcohol, cetostearyl alcohol, behenyl alcohol, oleyl alcohol, 2-heptyl-1-undecanol, 1,17-hepatadecanediol and mixtures thereof.

In another aspect of the present application, the skin penetration enhancer is branched chain fatty alcohol which is selected from 2-methyl-1-pentanol, 2-ethyl-hexanol, 2-propyl-heptanol, 2-butyl-octanol, 2-pentyl-1-nonanol, 2-hexyl-1-decanol, 1,6-hexanediol, 1,7-heptanediol, 1,8-octanediol, 1,9-nonanediol, 1,10-decanediol, 1,11-undecanediol, 1,12-dodecanediol, 1,13-tridecanediol, 1,14-tetradecanediol, 1,15-pentadecanediol, 1,16-hexadecanediol, 1,17-heptadecanediol, 1,18-octadecanediol and mixtures thereof.

In another aspect, the skin penetration enhancer is selected from unsaturated fatty alcohols.

In another aspect, the skin penetration enhancer is selected from unsaturated fatty alcohol having at least one unsaturation bond in the fatty alcohol chain and having “Z” configuration. In another aspect, oleyl alcohol is a skin penetration enhancer in the context of present application.

In another aspect, composition of the present application comprises one or more additional active agents useful in the management of psoriasis and associated pathological conditions including synthetic, semi-synthetic, or naturally obtained active agents. The compositions of the present application can be used for prophylaxis, amelioration, or treatment of skin diseases and disorders, by administration of a pharmaceutically and/or dermatologically effective amount of the spray composition to a subject in need thereof. The compositions of the present application are also useful in conjunction with other therapies, such as phototherapy.

In another aspect, the composition of the present application is easily water-washable and removable from the site of application.

In another aspect, the composition of the present application, when applied by spraying onto the skin, are substantially non-occlusive to the skin and does not form any film layer/residues at the site of application.

In another aspect, the compositions of the present application are substantially free of propylene glycol.

In another aspect, the composition of the present application is substantially free of (C₁-C₄) alcohols and/or propylene glycol, such that any amounts present do not cause significant skin irritation or impart any undesired attributes to the composition.

In another aspect, the composition of the present application is substantially non-foaming, free of propylene glycol and free of propellant.

In another aspect, the composition of the present application is substantially free of glycols. The glycols according to the present application are alkylene or polyalkylene glycols. Examples include (C₁ to C₆) alkylene and polyalkylene glycols, such as ethylene glycol, polyethylene glycol (2 to 20 monomers), propylene glycol, dipropylene glycol, butylene glycol, pentylene glycol and hexylene glycol. They may or may not be oxyethylenated (2 to 50 EO). Also exemplary are glycol ethers, such as ethoxydiglycol or diethylene glycol monoethyl ether, marketed under the trademark Transcutol HP by Gattefosse, propylene glycol laurate marketed under the trademark Lauroglycol by Gattefosse, propylene glycol dicaprate dicaprylate marketed under the trademark Estol 1526 by Uniqema, and propylene glycol dipelargonate.

In another aspect, the composition of the present application does not cause significant skin irritation even in occlusive condition. An aqueous based topical spray composition of the present application is substantially free of propylene glycol and stable for at least for the period of about 6 months at 40° C. or at least for the period of 24 months at 25° C.

In another aspect, the aqueous based topical spray composition is stable for at least for the period of about 6 months at 40° C. or at least for the period of 24 months at 25° C.

In an aspect, the aqueous based topical spray composition comprising: a) a betamethasone compound; b) oleyl alcohol; c) at least one emulsifying agent; d) at least one pharmaceutically and acceptable excipient; and water; wherein said composition is skin depot composition and is stable for at least about 6 months at 40° C. or at least for the period of 24 months at 25° C.

In another aspect, the composition of the present application does not form film at application site.

In another aspect, an aqueous based topical spray composition of present application can be administered for more than 1, 2, 3, 4, 5, 6, 7, 8, 9, 10, 11, 12, 13, 14, 15, 16, 17, 18, 19, 20, 21, 22, 23, 24, 25, 26, 27, or 28 days based on severity of the disease condition with substantially no hypothalamic-pituitary-adrenal (HPA) axis suppression.

In another aspect, an aqueous based topical spray composition of present application can be administered for up to days based on severity of the disease condition with substantially no hypothalamic-pituitary-adrenal (HPA) axis suppression.

In some embodiments the method comprises administering an aqueous-based topical spray composition comprising a betamethasone compound once or twice daily to an affected area of skin of a subject, wherein the composition is administered up to days based on severity of the disease condition with substantially no hypothalamic-pituitary-adrenal (HPA) axis suppression.

One embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, once or twice daily to the affected area of skin of the subject, wherein the composition is administered at least for a day, which provides HPA-axis suppression lower or at par with Diprolene lotion, 0.05%.

One embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, once or twice daily to the affected area of skin of the subject, wherein the composition is administered at least for up to 15 days, which provides HPA-axis suppression lower or at par with Diprolene lotion, 0.05%.

One embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, once or twice daily to the affected area of skin of the subject, wherein the composition is administered at least for up to 29 days, which provides HPA-axis suppression lower or at par with Diprolene lotion, 0.05%.

Another embodiment of the present application relates to a method of treating skin diseases or disorders such as psoriasis, steroid responsive dermatoses, erythema, contact sensitivity reactions, and other associated diseases or disorders comprising administration of topical spray composition comprising betamethasone compound, once or twice daily to the affected area of skin of the subject, wherein the composition can be administered up to 29 days based on severity of the disease condition with substantially no HPA-axis suppression.

In one aspect of the present application, the skin disease is moderate to severe plaque psoriasis.

In one aspect of the present application, the skin disease is moderate plaque psoriasis.

Another aspect of the present application provides dispensing devices for the topical delivery of the compositions onto the skin in the form of sprays. In another aspect, the present application provides devices, into which the composition is filled, comprising a container, a dispenser, and a closure.

Another aspect of the present application relates to a dispensing device containing propellant-free topical composition, wherein a device comprises a container, a pump dispenser, a dip tube, a metering valve, and an actuator, and wherein the pump dispenser is capable of dispensing the composition through a dip tube into a metering valve, and through the actuator fitted with an orifice, such that the composition is consistently released in the form of a substantially uniform spray.

An aspect of the present application relates to dispensing device containing a propellant-free topical composition, wherein the device comprises a container having therein a pouch system or bag filled with the composition, optionally fitted with a dip tube and an actuator fitted with a valve, the container being filled with a gas such as nitrogen gas or compressed air, surrounding the pouch or bag. Introduction of the composition into the system can further increase the pressure of the system, which is capable of dispensing the composition from the pouch or bag into the actuator fitted with a valve, such that the composition is released upon actuation in the form of a spray.

In another aspect, advantages of topical sprayable composition of the present application include non-irritancy to the site of application, ease of application, usefulness for long periods, non-staining of fabrics, and not possessing a strong or objectionable odor. This facilitates a subject in need thereof to maintain regular applications of the medications, and thus avoids abrupt withdrawal of the corticosteroid composition application, which in turn prevents an aggressive recurrence of the disease condition.

In another embodiment, the topical spray composition of the present application comprising betamethasone compound provides ‘mean Cmax’ of betamethasone-17, 21-dipropionate, betamethasone-17-propionate and betamethasone base (sum of ‘mean Cmax’ values of individual products) of less than about 400 pg/ml, when administered twice daily to a subject for 15 days. In another aspect ‘mean Cmax’ is in the range of about 10 pg/ml to about 300 pg/ml. In one aspect of the above embodiment, ‘mean Cmax’ is in the range of about 10 pg/ml to about 275 pg/ml.

In another embodiment, the topical spray composition of the present application comprising betamethasone compound provides ‘mean Cmax’ of betamethasone-17, 21-dipropionate, betamethasone-17-propionate and betamethasone base (sum of ‘mean Cmax’ values of individual products) of less than about 400 pg/ml, when administered twice daily to a subject for 29 days. In another aspect ‘mean Cmax’ is in the range of about 10 pg/ml to about 300 pg/ml. In one aspect of the above embodiment, ‘mean Cmax’ is in the range of about 50 pg/ml to about 290 pg/ml.

In another embodiment, the topical spray composition of the present application comprising betamethasone compound provides ‘mean Cmax’ of betamethasone base of less than about 300 pg/ml, when administered twice daily to a subject for 15 days. In one aspect of the above embodiment, ‘mean Cmax’ is in the range of about 20 pg/ml to about 250 pg/ml. In another aspect, ‘mean Cmax’ is in the range of about 50 pg/ml to about 200 pg/ml.

In another embodiment, the topical spray composition of the present application comprising betamethasone compound provides ‘mean Cmax’ of betamethasone base of less than about 150 pg/ml, when administered twice daily to a subject for 29 days. In one aspect of the above embodiment, ‘mean Cmax’ is in the range of about 5 pg/ml to about 100 pg/ml. In another aspect, ‘mean Cmax’ is in the range of about 20 pg/ml to about 90 pg/ml.

In another embodiment, the topical spray composition of the present application comprising betamethasone compound provides ‘mean Cmax’ of betamethasone-17-propionate of less than about 300 pg/ml, when administered twice daily to a subject for 15 days. In one aspect of the above embodiment, ‘mean Cmax’ is in the range of about 20 pg/ml to about 250 pg/ml. In another aspect, ‘mean Cmax’ is in the range of about 50 pg/ml to about 200 pg/ml.

In another embodiment, the topical spray composition of the present application comprising betamethasone compound provides ‘mean Cmax’ of betamethasone-17-propionate of less than about 200 pg/ml, when administered twice daily to a subject for 29 days. In one aspect of the above embodiment, ‘mean Cmax’ is in the range of about 10 pg/ml to about 150 pg/ml. In another aspect, ‘mean Cmax’ is in the range of about 30 pg/ml to about 125 pg/ml.

In another embodiment, the topical spray composition of the present application comprising betamethasone compound provides ‘mean Cmax’ of betamethasone-17, 21-dipropionate of less than about 100 pg/ml, when administered twice daily to a subject for 15 days. In one aspect of the above embodiment, ‘mean Cmax’ is in the range of about 5 pg/ml to about 75 pg/ml. In another aspect, ‘mean Cmax’ is in the range of about 5 pg/ml to about 50 pg/ml. In further aspect, ‘mean Cmax’ is in the range of 5 pg/ml to about 30 pg/ml. In another aspect, ‘mean Cmax’ is not measurable (<5 pg/ml)

In another embodiment, the topical spray composition of the present application comprising betamethasone compound provides a ‘Cmax’ of betamethasone base of less than about 1000 pg/ml, when administered twice daily to a subject for 15 days. In one aspect, Cmax of less than about 900 pg/ml. In another aspect, ‘Cmax’ is in the range of about 5 pg/ml to about 850 pg/ml. In another aspect, ‘Cmax’ is in the range of about 5 pg/ml to about 800 pg/ml.

In another embodiment, the topical spray composition of the present application comprising betamethasone compound provides a ‘Cmax’ of betamethasone base of less than about 500 pg/ml, when administered twice daily to a subject for 29 days. In one aspect, Cmax of less than about 400 pg/ml. In another aspect, ‘Cmax’ is in the range of about 5 pg/ml to about 400 pg/ml. In another aspect, ‘Cmax’ is in the range of about 5 pg/ml to about 300 pg/ml. In another aspect, ‘Cmax’ is in the range of about 5 pg/ml to about 250 pg/ml.

In another embodiment, the topical spray composition of the present application comprising betamethasone compound provides a ‘Cmax’ of betamethasone-17-propionate of less than about 700 pg/ml, when administered twice daily to a subject for 15 days. In one aspect, ‘Cmax’ is in the range of about 5 pg/ml to about 600 pg/ml. In another aspect, ‘Cmax’ is in the range of about 6 pg/ml to about 550 pg/ml.

In another embodiment, the topical spray composition of the present application comprising betamethasone compound provides a ‘Cmax’ of betamethasone-17-propionate of less than about 500 pg/ml, when administered twice daily to a subject for 29 days. In one aspect, ‘Cmax’ is in the range of about 5 pg/ml to about 400 pg/ml. In another aspect, ‘Cmax’ is in the range of about 6 pg/ml to about 350 pg/ml.

In another embodiment, the topical spray composition of the present application comprising betamethasone compound provides a ‘Cmax’ of betamethasone-17, 21-dipropionate of less than about 100 pg/ml, when administered twice daily to a subject for 15 days. In one aspect, ‘Cmax’ of less than about 75 pg/ml. In another aspect, ‘Cmax’ is in the range of about 5 pg/ml to about 75 pg/ml. In another aspect, ‘Cmax’ is not measurable (<5 pg/ml).

In another embodiment, the topical spray composition of the present application comprising betamethasone compound provides ‘median Cmax’ of betamethasone base of less than about 100 pg/ml, when administered twice daily to a subject for 15 days. In one aspect of the above embodiment, ‘median Cmax’ is in the range of about 20 pg/ml to about 80 pg/ml. In another aspect, ‘median Cmax’ is in the range of about 20 pg/ml to about 65 pg/ml.

In another embodiment, the topical spray composition of the present application comprising betamethasone compound provides ‘median Cmax’ of betamethasone base of less than about 100 pg/ml, when administered twice daily to a subject for 29 days. In one aspect of the above embodiment, ‘median Cmax’ is in the range of about 15 pg/ml to about 75 pg/ml. In another aspect, ‘median Cmax’ is in the range of about 20 pg/ml to about 65 pg/ml.

In another embodiment, the topical spray composition of the present application comprising betamethasone compound provides ‘median Cmax’ of betamethasone-17-propionate of less than about 200 pg/ml, when administered twice daily to a subject for 15 days. In one aspect of the above embodiment, ‘median Cmax’ is in the range of about 10 pg/ml to about 150 pg/ml. In another aspect, ‘median Cmax’ is in the range of about 20 pg/ml to about 100 pg/ml.

In another embodiment, the topical spray composition of the present application comprising betamethasone compound provides ‘median Cmax’ of betamethasone-17-propionate of less than about 200 pg/ml, when administered twice daily to a subject for 29 days. In one aspect of the above embodiment, ‘median Cmax’ is in the range of about 10 pg/ml to about 150 pg/ml. In another aspect, ‘median Cmax’ is in the range of about 20 pg/ml to about 100 pg/ml.

In another embodiment, the topical spray composition of the present application comprising betamethasone compound provides lower ‘mean Cmax’ of betamethasone base when administered to a subject for 29 days compared to that of twice-daily administration for 15 days.

In another embodiment, the topical spray composition of the present application comprising betamethasone compound provides lower ‘median Cmax’ of betamethasone-17-propionate when compared to that of Diprolene lotion, 0.05%, when administered twice daily to a subject for 15 days.

In another embodiment, the topical spray composition of the present application comprising betamethasone compound provides lower ‘median Cmax’ of betamethasone-17, 21-dipropionate when compared to that of Diprolene lotion, 0.05%, when administered twice daily to a subject for 15 days.

In another embodiment, the topical spray composition of the present application comprising betamethasone compound provides lower ‘median Cmax’ of betamethasone base when compared to that of Diprolene lotion, 0.05%, when administered twice daily to a subject for 15 days.

In another embodiment, the topical spray composition of the present application comprising betamethasone compound provides HPA axis suppression no greater when administered twice-a-day for 15 days, compared with Diprolene lotion, 0.05% administered twice-a-day for 15 days in subjects with moderate to severe plaque psoriasis under maximal use conditions.

In another embodiment, the topical spray composition of the present application comprising betamethasone compound provides HPA axis suppression no greater when administered twice-a-day for 29 days, compared with Diprolene lotion, 0.05% administered twice-a-day for 15 days in subjects with moderate to severe plaque psoriasis under maximal use conditions.

One embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides ‘mean Cmax’ of betamethasone-17, 21-dipropionate, betamethasone-17-propionate and betamethasone base (sum of ‘mean Cmax’ values of individual products) of less than about 400 pg/ml, when administered twice daily to a subject for 15 days. In another aspect ‘mean Cmax’ is in the range of about 10 pg/ml to about 300 pg/ml. In one aspect of the above embodiment, ‘mean Cmax’ is in the range of about 10 pg/ml to about 275 pg/ml.

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides ‘mean Cmax’ of betamethasone-17, 21-dipropionate, betamethasone-17-propionate and betamethasone base (sum of ‘mean Cmax’ values of individual products) of less than about 400 pg/ml, when administered twice daily to a subject for 29 days. In another aspect ‘mean Cmax’ is in the range of about 10 pg/ml to about 300 pg/ml. In one aspect of the above embodiment, ‘mean Cmax’ is in the range of about 50 pg/ml to about 290 pg/ml.

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides ‘mean Cmax’ of betamethasone base of less than about 300 pg/ml, when administered twice daily to a subject for 15 days. In one aspect of the above embodiment, ‘mean Cmax’ is in the range of about 20 pg/ml to about 250 pg/ml. In another aspect, ‘mean Cmax’ is in the range of about 50 pg/ml to about 200 pg/ml.

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides ‘mean Cmax’ of betamethasone base of less than about 150 pg/ml, when administered twice daily to a subject for 29 days. In one aspect of the above embodiment, ‘mean Cmax’ is in the range of about 5 pg/ml to about 100 pg/ml. In another aspect, ‘mean Cmax’ is in the range of about 20 pg/ml to about 90 pg/ml.

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides ‘mean Cₘₐₓ’ of betamethasone-17-propionate of less than about 300 pg/ml, when administered twice daily to a subject for 15 days. In one aspect of the above embodiment, ‘mean Cmax’ is in the range of about 20 pg/ml to about 250 pg/ml. In another aspect, ‘mean Cmax’ is in the range of about 50 pg/ml to about 200 pg/ml.

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides ‘mean Cmax’ of betamethasone-17-propionate of less than about 200 pg/ml, when administered twice daily to a subject for 29 days. In one aspect of the above embodiment, ‘mean Cmax’ is in the range of about 10 pg/ml to about 150 pg/ml. In another aspect, ‘mean Cmax’ is in the range of about 30 pg/ml to about 125 pg/ml.

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides ‘mean Cmax’ of betamethasone-17, 21-dipropionate of less than about 100 pg/ml, when administered twice daily to a subject for 15 days. In one aspect of the above embodiment, ‘mean Cmax’ is in the range of about 5 pg/ml to about 75 pg/ml. In another aspect, ‘mean Cmax’ is in the range of about 5 pg/ml to about 50 pg/ml. In further aspect, ‘mean Cmax’ is in the range of 5 pg/ml to about 30 pg/ml. In another aspect, ‘mean Cmax’ is not measurable (<5 pg/ml)

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides a ‘Cmax’ of betamethasone base of less than about 1000 pg/ml, when administered twice daily to a subject for 15 days. In one aspect, ‘Cmax’ of less than about 900 pg/ml. In another aspect, ‘Cmax’ is in the range of about 5 pg/ml to about 850 pg/ml. In another aspect, ‘Cmax’ is in the range of about 5 pg/ml to about 800 pg/ml.

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides a ‘Cmax’ of betamethasone base of less than about 500 pg/ml, when administered twice daily to a subject for 29 days. In one aspect, Cmax of less than about 400 pg/ml. In another aspect, ‘Cmax’ is in the range of about 5 pg/ml to about 400 pg/ml. In another aspect, ‘Cmax’ is in the range of about 5 pg/ml to about 300 pg/ml. In another aspect, ‘Cmax’ is in the range of about 5 pg/ml to about 250 pg/ml.

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides a ‘Cmax’ of betamethasone-17-propionate of less than about 700 pg/ml, when administered twice daily to a subject for 15 days. In one aspect, ‘Cmax’ is in the range of about 5 pg/ml to about 600 pg/ml. In another aspect, ‘Cmax’ is in the range of about 6 pg/ml to about 550 pg/ml.

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides a ‘Cmax’ of betamethasone-17-propionate of less than about 500 pg/ml, when administered twice daily to a subject for 29 days. In one aspect, ‘Cmax’ is in the range of about 5 pg/ml to about 400 pg/ml. In another aspect, ‘Cmax’ is in the range of about 6 pg/ml to about 350 pg/ml.

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides a ‘Cmax’ of betamethasone-17, 21-dipropionate of less than about 100 pg/ml, when administered twice daily to a subject for 15 days. In one aspect, ‘Cmax’ is less than about 75 pg/ml. In another aspect, ‘Cmax’ is in the range of about 5 pg/ml to about 75 pg/ml. In another aspect, ‘Cmax’ is not measurable (<5 pg/ml)

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides ‘median Cmax’ of betamethasone base of less than about 100 pg/ml, when administered twice daily to a subject for 15 days. In one aspect of the above embodiment, ‘median Cmax’ is in the range of about 20 pg/ml to about 80 pg/ml. In another aspect, ‘median Cmax’ is in the range of about 20 pg/ml to about 65 pg/ml.

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides ‘median Cmax’ of betamethasone base of less than about 100 pg/ml, when administered twice daily to a subject for 29 days. In one aspect of the above embodiment, ‘median Cmax’ is in the range of about 15 pg/ml to about 75 pg/ml. In another aspect, ‘median Cmax’ is in the range of about 20 pg/ml to about 65 pg/ml.

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides ‘median Cmax’ of betamethasone-17-propionate of less than about 200 pg/ml, when administered twice daily to a subject for 15 days. In one aspect of the above embodiment, ‘median Cmax’ is in the range of about 10 pg/ml to about 150 pg/ml. In another aspect, ‘median Cmax’ is in the range of about 20 pg/ml to about 100 pg/ml.

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides ‘median Cmax’ of betamethasone-17-propionate of less than about 200 pg/ml, when administered twice daily to a subject for 29 days. In one aspect of the above embodiment, ‘median Cmax’ is in the range of about 10 pg/ml to about 150 pg/ml. In another aspect, ‘median Cmax’ is in the range of about 20 pg/ml to about 100 pg/ml.

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides lower ‘mean Cmax’ of betamethasone base when administered to a subject for 29 days compared to that of twice-daily administration for 15 days.

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides lower ‘median Cmax’ of betamethasone-17-propionate when compared to that of Diprolene lotion, 0.05%, when administered twice daily to a subject for 15 days.

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides lower ‘median Cₘₐₓ’ of betamethasone-17, 21-dipropionate when compared to that of Diprolene lotion, 0.05%, when administered twice daily to a subject for 15 days.

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides lower ‘median Cmax’ of betamethasone base when compared to that of Diprolene lotion, 0.05%, when administered twice daily to a subject for 15 days.

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides HPA axis suppression no greater when administered twice-a-day for 15 days, compared with Diprolene lotion, 0.05% administered twice-a-day for 15 days in subjects with moderate to severe plaque psoriasis under maximal use conditions.

Another embodiment of the present application relates to a method of treating skin diseases or disorders such as psoriasis, steroid responsive dermatoses, erythema, contact sensitivity reactions, and other associated diseases or disorders, comprising administration of topical spray composition comprising betamethasone compound, which provides HPA axis suppression no greater when administered twice-a-day for 29 days, compared with Diprolene lotion, 0.05% administered twice-a-day for 15 days in subjects with moderate to severe plaque psoriasis under maximal use conditions.

In one aspect of the present application, the skin disease is moderate to severe plaque psoriasis.

In one aspect of the present application, the skin disease is moderate plaque psoriasis.

In one aspect of the above embodiments, the betamethasone compound is betamethasone dipropionate.

In one aspect of the above embodiments, the betamethasone compound is betamethasone dipropionate.

In another aspect, a corticosteroid present in the topical compositions is betamethasone dipropionate, which typically is administered in doses of about 0.001 mg/kg body weight to about 0.5 mg/kg body weight, to a subject in need thereof.

In another aspect, the compositions of the present application may be in the form of solutions, suspensions, emulsions, lotions, microemulsions, nanoemulsions, emulgels, gels, and the like. In embodiments, compositions may be in the form of an emulsion. The emulsion can be in the form of an oil-in-water type of emulsion or a water-in-oil type of emulsion. An aqueous-based emulsion, such as an oil-in-water emulsion, frequently has lower viscosity than other emulsion types and exhibits appreciable storage stability. Generally, oil-in-water emulsions have better skin feel properties, when applied to the skin, as these give sensations similar to an aqueous material. When the oily phase is dispersed as droplets within an aqueous continuous phase, this is called an “oil-in-water” type of emulsion. When an aqueous phase is dispersed as droplets within an oily continuous phase, this is called a “water-in-oil” type of emulsion. In an aspect, the hydrophobic phase comprises about 0.5% to about 90% by weight of the composition. Compositions in the form of emulsions may be micro- or nano-emulsions. In embodiments, average size of the dispersed phase droplets are less than about 500 μm. In an aspect, average size of the dispersed phase droplets are less than about 2000 nm. In an aspect, D90 of the dispersed phase droplets is in the range of about 1 μm to about 10 μm.

In another aspect, the compositions of the present application are formulated as emulsions, comprising an oily or hydrophobic phase, an aqueous or hydrophilic phase, and an emulsifier.

In another aspect, composition of the present application include pharmaceutically and/or dermatologically acceptable excipients including, but not limited to, one or more of carriers, emulsifiers, coemulsifiers, permeation or penetration enhancers, solvents, co-solvents, emollients, antioxidants, preservatives, buffering agents, gelling or thickening agents, polymers, surfactants, soothing agents, pH modifiers, solubilizers, humectants, emollients, moisturizers, oily bases, and the like.

Examples of suitable polymers for use in the present application include, but are not limited to carbomers, polyethylene glycols, acrylate polymers, methacrylate polymers, polyvinylpyrrolidones, copolymers based on butyl methacrylate and methyl methacrylate povidone, vinyl acetates, polyvinyl acetates, celluloses, gums, alginates, cellulose acetate phthalates, cellulose acetate butyrates, hydroxypropyl methyl cellulose phthalates, and the like. Examples include Carbopol® products, PEG 400, Eudragit® 100, Eudragit® RSPO, Eudragit® RLPO, Eudragit® ND40, Plasdone®, copolymers based on butyl methacrylate and methyl methacrylate (Plastoid® B), alkyl celluloses such as ethyl celluloses and methyl celluloses, hydroxyalkyl celluloses such as hydroxyethyl cellulose and hydroxypropyl cellulose, hydroxyalkyl alkyl celluloses such as hydroxypropyl methylcelluloses and hydroxybutyl methylcelluloses, gums such as xanthan gum, tragacanth, guar gum, locust bean gum, acacia, and the like.

Other polymers that are useful include polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyglycolides, polysiloxanes, polyurethanes and copolymers thereof, cellulose ethers, cellulose esters, nitrocelluloses, polymers of acrylic and methacrylic esters, cellulose acetates, cellulose propionates, cellulose acetate butyrates, cellulose acetate phthalates, carboxylethyl celluloses, cellulose triacetates, cellulose sulphate sodium salts, poly(methyl ethacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylenes, polypropylenes, poly(ethylene glycol), poly(ethylene oxide), poly(ethylene terephthalate), poly(vinyl alcohol), poly(vinyl acetate), poly(vinyl chloride), polystyrenes, and the like, including any mixtures thereof.

Further useful polymers include synthetic polymers, such as polymers of lactic acid and glycolic acid, polyanhydrides, poly(ortho ester), polyurethanes, poly(butyric acid), poly(valeric acid), poly(caprolactone), poly(hydroxybutyrate), poly(lactide-co-glycolide), poly(lactide-co-caprolactone), and natural polymers such as alginate and other polysaccharides that include but are not limited to arabinans, fructans, fucans, galactans, galacturonans, glucans, mannans, xylans (such as, for example, inulin), levan, fucoidan, carrageenan, galatocarolose, pectic acid, pectin, amylose, pullulan, glycogen, amylopectin, cellulose, dextran, pustulan, chitin, agarose, keratan, chondroitan, dermatan, hyaluronic acid, alginic acid, xanthan gum, starches, and various other natural homopolymers and heteropolymers, such as those containing one or more of aldoses, ketoses, acids or amines, erythrose, threose, ribose, arabinose, xylose, lyxose, allose, altrose, glucose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof, and including dextran and cellulose, collagen, albumin and other hydrophilic proteins, zein and other prolamines and hydrophobic proteins, copolymers or mixtures thereof.

In further aspects, the amount of polymer is in the range of from about 0.001% w/w to about 45% w/w of the total weight of the composition. In an aspect, the amount of polymer is in the range of from about 0.01% w/w to about 5% w/w of the total weight of the composition. In an aspect, the amount of polymer is in the range of from about 0.1% w/w based on total weight of the composition. In an aspect, the amount of polymer of less than about 0.1% w/w based on total weight of the composition. In an aspect, the amount of polymer is about 0.05% w/w based on total weight of the composition.

Examples of suitable emulsifying agents include, but are not limited to, disodium cocoampho diacetate, oxyethylenated glyceryl cocoate (7 EO), PEG 30 Dipolyhydroxy stearate (Cithrol DPHS), Polyglyceryl-3 Diisostearate, PEG-20 hexadecenyl succinate, PEG-15 stearyl ether, Polyoxyl 20 Cetostearyl Ether, Polypropylene Glycol (PPG)-Stearyl Ether such as PPG-11 Stearyl Ether and PPG-15 Stearyl Ether, Polyoxypropylene Stearyl Ether (Arlamol E), ricinoleic monoethanolamide monosulfosuccinate salts, oxyethylenated hydrogenated ricinoleic triglyceride containing 60 ethylene oxide units such as the products sold by BASF under the trademarks Cremophor® RH 60 or Cremophor® RH 40 (polyoxyl 40 hydrogenated castor oil), polymers such as poloxamers, which are block copolymers of ethylene oxide and propylene oxide, and the nonsolid fatty substances at room temperature (that is to say, at temperatures ranging from about to 35° C.) such as sesame oil, sweet almond oil, apricot stone oil, sunflower oil, octoxyglyceryl palmitate (or 2-ethylhexyl glyceryl ether palmitate), octoxyglyceryl behenate (or 2-ethylhexyl glyceryl ether behenate), dioctyl adipate, and tartrates of branched dialcohols. Sorbitan fatty acid esters are a series of mixtures of partial esters of sorbitol and its mono- and dianhydrides with fatty acids. Sorbitan esters include products sold as Arlacel® 20, Arlacel 40, Arlacel 60, Arlacel 80, Arlacel 83, Arlacel 85, Arlacel 987, Arlacel C, PEG-6 stearate and glycol stearate and PEG-32 stearate (Tefose® 63), and PEG-6 stearate and PEG-32 stearate (Tefose® 1500), and any mixtures thereof. Polyethylene glycol ethers of stearic acid are in another group of emulsifiers that can be used in the emulsions. Examples of polyethylene glycol ethers of stearic acid are steareth-2, steareth-4, steareth-6, steareth-7, steareth-10, steareth-11, steareth-13, steareth-15, steareth-20, polyethylene glycol ethers of stearyl alcohol (steareth 21), and any mixtures thereof. Other emulsifying agents include sodium lauryl sulphate, cetyl trialkyl ammonium bromide, polyoxyethylene sorbitan fatty acid esters or any mixtures thereof.

In an aspect, the emulsifying agent is selected from nonionic surfactant.

Nonionic emulsifying agents include those that can be broadly defined as condensation products of long chain alcohols, e.g., C₈₋₃₀ alcohols, with sugar or starch polymers, i.e., glycosides. Various sugars include, but are not limited to, glucose, fructose, mannose, and galactose, and various long chain alcohols include, but are not limited to, decyl alcohol, cetyl alcohol, stearyl alcohol, lauryl alcohol, myristyl alcohol, oleyl alcohol, and the like.

Other useful nonionic emulsifying agents include condensation products of alkylene oxides with fatty acids such as alkylene oxide esters of fatty acids. Other nonionic surfactants are the condensation products of alkylene oxides with two moles of fatty acids such as alkylene oxide diesters of fatty acids.

Emulsifying agents can also include any of a wide variety of cationic, anionic, zwitterionic, amphoteric and nonionic surfactants and combinations thereof, which are known in the art. Non-limiting examples of anionic emulsifying agents include alkyl is ethionates, alkyl and alkyl ether sulfates and salts thereof, alkyl and alkyl ether phosphates and salts thereof, alkyl methyl taurates, and soaps (e.g., alkali metal salts and sodium or potassium salts) of fatty acids.

Examples of amphoteric and zwitterionic emulsifying agents include those which are broadly described as derivatives of aliphatic secondary and tertiary amines in which the aliphatic radical can be straight or branched chain, wherein one of the aliphatic substituents contains from about 8 to about 22 carbon atoms and one contains an anionic water solubilizing group, e.g., carboxy, sulfonate, sulfate, phosphate, or phosphonate. Specific examples include alkylimino acetates, iminodialkanoates and aminoalkanoates, imidazolinium and ammonium derivatives. Other suitable amphoteric and zwitterionic emulsifying agents include betaines, sultaines, hydroxysultaines, alkyl sarcosinates, and alkanoyl sarcosinates.

Silicone emulsifying agents are typically organically modified organopoly siloxanes, sometimes called silicone surfactants. Useful silicone emulsifying agents include dimethicone copolyols. These materials are polydimethyl siloxanes, which have been modified to include polyether side chains such as polyethylene oxide chains, polypropylene oxide chains, mixtures of these chains, and polyether chains containing moieties derived from both ethylene oxide and propylene oxide.

The amounts of emulsifying agent are from about 0.25% to about 45% of the total weight of the composition.

Co-emulsifiers or secondary emulsifying agents include polyoxylglycerides such as oleoyl macrogolglycerides (Labrafil® M 1944CS), linoleoyl macrogolglycerides (Labrafil® M2125CS), caprylocaproyl macrogolglycerides (Labrasol®), cetyl alcohol (and) ceteth-20 (and) steareth-20 (Emulcire™ 61 WL 2659), glyceryl stearate (and) PEG-75 stearate (Gelot® 64), or any mixtures thereof.

In an aspect, the emulsifying agents of the present application may act as skin penetration enhancers.

In an aspect, the composition further comprises one or more antioxidant, preservative, humectant, or plasticizer.

Antioxidants are substances which inhibit oxidation or suppress reactions promoted by oxygen or peroxides. Antioxidants, especially lipid-soluble antioxidants, can be absorbed into the cellular membrane to neutralize oxygen radicals and thereby protect the membrane. Suitable antioxidants for compositions of the present application include, but are not limited to, ascorbic acid (vitamin C), glutathione, lipoic acid, uric acid, carotenes, a-tocopherol (vitamin E), ubiquinol, butylated hydroxyanisole, butylated hydroxytoluene, sodium benzoate, propyl gallate (PG, E310), and tertiary-butylhydroquinone. The amounts of antioxidant are from about 0.01% to about 20%, of the total weight of the composition.

Some of the excipient substances described above can have more than one function in a formulation. For example, a substance can be both a solvent and a penetration enhancer, or both a solvent and a carrier. The categorizations of materials described above are not to be construed as limiting or restricting in any manner.

The compositions can be applied directly onto affected areas of the skin, such as psoriatic plaques or dermatoses. Sprayable compositions, upon being sprayed, form droplets/mist on the affected areas and, in embodiments, can provide release of the active agent for an extended duration of time.

Addition of fatty alcohol may lead the sprayable composition may build up more viscosity however an aqueous based topical spray composition of the present application is low viscos and sprayable composition and an aqueous based topical spray composition of the present application comprises at least one fatty alcohols in the range of about 5% w/w based on total weight of the composition. Viscosities of aqueous-based emulsions of the present application frequently vary in the range of about 0.01-15 Pascal second, “Pa·s” (10-15,000 centipoise, “cP”), about 0.1-1.5 Pa·s (100-1,500 cP), or about 0.2-1 Pa·s (200-1,000 cP). In an aspect, the topical spray composition of the present application having pourable liquid like consistency and viscosity from about 100 cP to about 1000 cP when measured by Brookfield viscometer DVII+Pro, spindle LV3 at 100 rpm.

The topical spray compositions of the present application comprising: a) at least one betamethasone compound; b) at least one fatty alcohol selected from group comprising of elaidyl alcohol, linoleyl alcohol, linolenyl alcohol, caproic alcohol, lauryl alcohol, stearyl alcohol, cetostearyl alcohol, behenyl alcohol, oleyl alcohol, 2-heptyl-1-undecanol, 1,17-hepatadecanediol, and mixtures thereof; c) at least one emulsifying agent; d) at least one pharmaceutically and/or dermatologically acceptable excipient; and e) water; wherein said composition provides more retention of betamethasones in skin layers and low percentages of betamethasones permeated into receptor level in the In vitro percutaneous absorption and penetration study as described in Example 3.

In another aspect, the topical spray composition of the present application provides below about 10% of betamethasones permeated through receptor level (of the applied dose) in the In vitro percutaneous absorption and penetration study as described in Example 3.

In another aspect, the percentage of betamethasones permeated into the receptor level of less than about 5% of applied dose.

In another aspect, the composition of the present application comprising: a) a betamethasone compound present in amounts equivalent to about 0.05 percent by weight of the total composition; b) oleyl alcohol; c) at least one emulsifying agent; d) at least one pharmaceutically and/or dermatologically acceptable excipient; and e) water; provides below about 2% of betamethasones permeated through receptor level (of the applied dose) in the In vitro percutaneous absorption and penetration study as described in Example 3.

In another aspect, the composition of the present application comprising: a) a betamethasone compound present in amounts equivalent to about 0.05 percent by weight of the total composition; b) oleyl alcohol; c) at least one emulsifying agent; d) at least one pharmaceutically and/or dermatologically acceptable excipient; and e) water; provides between about 3 to about 8% of betamethasones retained in dermis and epidermis levels (of the applied dose) in the In vitro percutaneous absorption and penetration study as described in Example 3.

In another aspect, the topical spray composition of the present application provides output of from about 50 mg to about 230 mg per actuation or provides output of from about 160 mg to about 190 mg per actuation.

In another aspect, the topical spray composition of the present application provides retention of betamethasones in skin layers from about 0.1% to about 20% of applied dose in the In vitro percutaneous absorption and penetration study as described in Example 3.

In another aspect, the composition provides retention of betamethasones in skin layers from about 0.1% to about 10% of applied dose in the In vitro percutaneous absorption and penetration study as described in Example 3.

In another aspect, fatty alcohols used in topical composition provide higher skin layer retention of betamethasones and lower concentration of betamethasones permeated through receptor level. This tendency of the fatty alcohols provides skin depot compositions.

In another aspect, oleyl alcohol used in the composition of the present application provides skin retention ratio of about 50 in the In vitro percutaneous absorption and penetration study as described in Example 3. Further reference is made to Table 4 as set forth in Example 3.

The skin retention ratio is calculated using the below formula:

SKIN RETENTION RATIO=TOTAL BETAMETHASONES IN SKIN LAYERS/TOTAL BETAMETHASONES IN RECEPTOR.

In an aspect, the aqueous based topical spray composition of the present application is oil-in-water emulsion and having discontinuous oil phase and continuous aqueous phase.

In an aspect, the aqueous based topical spray composition of the present application, comprising: a) betamethasone compound; b) oleyl alcohol; c) at least one emulsifying agent; and d) water; wherein the composition is oil-in-water emulsion and comprising: a betamethasone compound in oil phase and the aqueous phase is substantially free of betamethasone compound.

In a specific aspect, the aqueous based topical spray composition of the present application, comprising: a) an oil phase comprising: i) betamethasone compound, ii) oleyl alcohol, and iii) at least one emulsifying agent; b) an aqueous comprising: water and c) at least one pharmaceutically and/or dermatologically acceptable excipient.

In an aspect, an aqueous based topical spray composition of the present application, comprising: a) a betamethasone dipropionate; b) oleyl alcohol; c) at least one emulsifying agent; and d) water; wherein the composition is oil-in-water emulsion and comprising the betamethasone dipropionate in the oil phase and the aqueous phase is substantially free of betamethasone dipropionate.

In an aspect, an aqueous based topical spray composition of the present application, comprising: a) a betamethasone dipropionate; b) an oil phase comprising: i) at least one fatty alcohol comprising: oleyl alcohol and ii) an emulsifying agent; and c) an aqueous phase comprising: water; wherein the composition comprising the betamethasone dipropionate in the oil phase and the aqueous phase is substantially free of betamethasone dipropionate.

In another aspect, the closure used for packaging are made of a polymeric substance such as high-density polyethylene (HDPE), low-density polyethylene (LDPE), or resins. The closures are particularly in the form of caps that are fitted onto the containers to aid in providing support to the dispenser unit and/or to shield the contents of the container from the outside environment. Various container materials include, but are not limited to, tin plated steel, aluminum, stainless steel, plastics, and glass.

An example of a dispenser is a unit containing a pump that can be adapted to fit on any type of container, such as by threads that match threading on the container or a self-locking joint whose mating parts exert a cam action, flexing until one part slips past a raised lip on the other part, preventing their separation. The pump is capable of dispensing sprayable compositions of the present application through a dip tube extending into a container from an actuator and attached to a one-way valve, which releases the composition from an orifice in the actuator in the form of a spray. The valve may be a metering valve.

Various types of valves that can be used include, but are not limited to, continuous spray valves and metering valves. The actuators allow for easy opening and closing of the valve and are an integral part of a package. This also serves to aid in producing the required type of product discharge. Various types of actuators include but are not limited to spray actuators, foam actuators, solid-stream actuators, and special actuators.

In another aspect, a dispensing device may be a device comprising a container, having therein a pouch system or bag containing the product, optionally fitted with a dip tube and an actuator fitted with a valve wherein the container is filled with nitrogen gas or compressed air, surrounding the pouch or bag. Containers can be made of aluminum or tin plate and the pouch system or bag containing the product can be made of layers of polyethylene (PE), polypropylene (PP), polyethylene terephthalate (PET), and aluminum. Introduction of the composition into the system further increases the pressure of the system which is capable of dispensing the composition from the pouch into the actuator, fitted with a valve, such that the composition is consistently released in the form of a substantially uniform spray upon actuation. The pouch can have a dip tube therein, communicating with the actuator valve, to control the spray rate and reduce droplet size.

In another aspect, a dispensing device useful for dispensing the compositions of the present application provides spray rates and spray patterns, in a manner such that substantially uniform dosage is dispensed each time which appreciably covers the desired affected area of the skin onto which the composition is sprayed. The pump is intended to deliver the composition uniformly onto the skin. It covers a desired area of the skin and produces very fine uniform droplets, at a specified spray rate such as, but not limited to, about 20 to about 500 mg/actuation, or about 100 to about 200 mg/actuation. The device provides a reproducible spray pattern, such as circular, frequently covering an area of about 0.1 to about 10 cm² depending on the distance from the application site.

About 2-6 priming actuations may be required for a new pump to reproducibly dispense the compositions. In a specific aspect, about 160 μL of a formulation is dispensed, per actuation of the pump. Devices frequently provide a reproducible distribution of droplets, in distributions where about 90% of the droplets have sizes ranging from about 1 to about 500 μm. The orifice is sized to control the droplet sizes of the dispensed product. The orifice size also affects providing of a uniform characteristic spray pattern.

In another aspect, the composition useful in treating psoriasis may be packaged in a bottle fitted with an attached spray pump closure that can be mechanically actuated by a subject or caregiver, to apply the composition to the affected skin (i.e., a pump-type spray closure).

In another aspect, a spray composition of the present application can be applied in an essentially easier and more exact way than creams and ointments can be applied. Using a spray application it is only necessary to spray a given volume, whereas the application of the semi-solid products (such as creams) requires an easily accessible and visual estimation of the cream amount or the ointment amount. Further, smearing and soiling of clothing can more easily be avoided on large surface areas using the spray compositions of the invention. For the spray compositions, spreading and rubbing are not necessary, contrary to cream and ointment products, since the layer formed on the body surface by evaporation or vaporization of the liquid already has an ideal fine dispersion of active agent; hence ‘pressure pain’ will not occur from the topical application of spray compositions of the present application.

In an aspect, an aqueous based emulsion sprayable composition of the present application also permit applying a medicament by a method whereby the area of application is contacted by only the spray (i.e.,) elbows, knees, scalp, and back. An aqueous based emulsion sprayable a composition of the present application is self-administered to area of application is contacted by only the spray (i.e.,) elbows, knees, scalp, and back.

In an aspect, the method of treating atopic dermatitis, seborrhoeic dermatitis, eczema, moderate to severe plaque psoriasis, rosacea, acne, steroid responsive dermatoses, erythema, contact sensitivity reactions and combinations thereof, the method comprising administering a pharmaceutically and/or dermatologically effective amount of topical spray composition directly onto an affected part of the skin of a subject in need thereof.

In an aspect, a method of treating atopic dermatitis, seborrhoeic dermatitis, eczema, moderate to severe plaque psoriasis, rosacea, acne, steroid responsive dermatoses, erythema, contact sensitivity reactions and combinations thereof comprising steps of: providing a device having a topical spray composition comprising: a betamethasone compound; and delivering a spray of said composition directly onto an affected part of the skin of a subject in need thereof, wherein the method provides spray characteristics of a wide angle full cone spray pattern having the first axis of from about 35 mm to about 60 mm, the second axis of from about 35 mm to about 55 mm, and the ratio between of first and second axis is from about 1 to about 1.5.

In an aspect, the administration distance is from about 20 mm to about 60 mm from subject's skin to device and the spray angle is from about 50 to about 70 degrees to the subject's skin.

In an aspect, the method of administering topical spray composition comprising steps of: providing a device having a topical spray composition comprising: a corticosteroid; and delivering a spray of said composition directly onto an affected part of the skin of a subject in need thereof, wherein the device delivers from about 65 mg to about 210 mg of spray composition per stroke, wherein the spray count from about 230 to about 270 strokes to empty the composition in the device.

In an aspect, topical application of compositions of the present application forms a depot on the skin without forming an occlusive film, thereby extending the duration of active agent action while allowing ‘breathing’ of the skin.

Another aspect of the present application further provide processes for preparing compositions that can be filled into suitable dispensing devices. In embodiments, processes comprise: a) preparing a composition comprising the active agent and one or more suitable excipients, and b) filling a desired quantity of the composition into a dispensing device.

In another aspect, process for preparing topical compositions comprising betamethasone compound as an active agent and excipients comprise: a) heating a mixture comprising an emulsifying agent and a solvent to obtain an oily phase; b) optionally, admixing an antioxidant and/or preservative into the oily phase of a); c) admixing a corticosteroid with a penetration enhancer; d) admixing the material of c) with material of a) or b); e) dissolving a polymer in an aqueous phase; f) admixing the oily phase of d) slowly with an aqueous phase of e) with continuous mixing; and g) homogenizing the mixture of f), followed by cooling.

In another aspect, betamethasone compound in the above process is selected from betamethasone dipropionate.

In another aspect, composition of the present application have pH values ranging from about 3 to about 7, or from about 3.5 to about 6.

In another aspect, the oil phase for an emulsion is a mixture of emulsifying agents and a solvent.

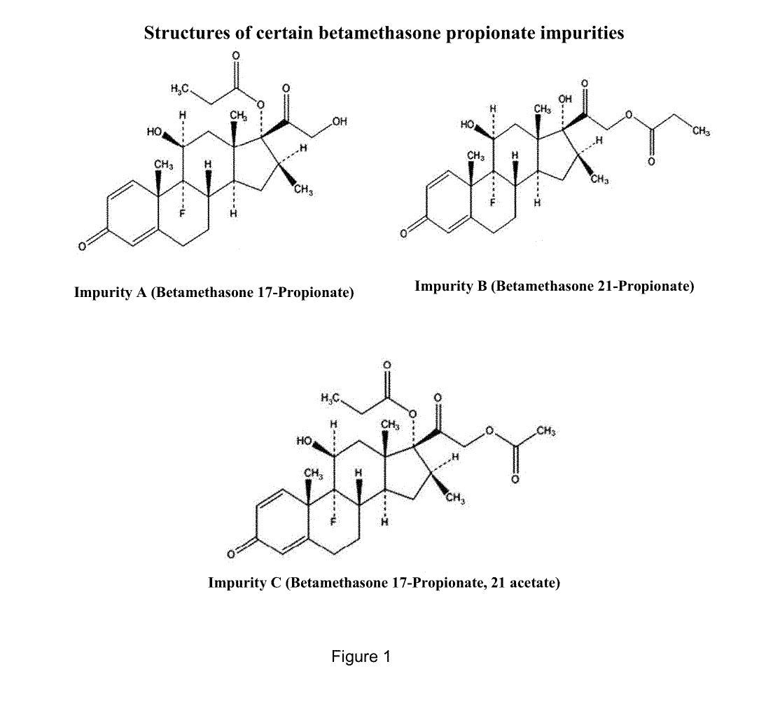

In another aspect, betamethasone propionate compositions of the present application may contain any one or more of impurities, such as impurity A (betamethasone-17-propionate) in amounts not more than about 5%, impurity B (betamethasone-21-propionate) in amounts not more than about 2%, impurity C (betamethasone-17-propionate 21-acetate) in amounts not more than about 1%, and single unknown impurity in amounts not more than about 1.0% (these impurities have the structures shown in FIG. 1), and any other drug-related impurities, in amounts such that any such impurities do not substantially adversely affect the safety of the composition. Impurities A and B are primarily observed during stability studies of a formulation, and impurity C is generally a process-related impurity from synthesis of the drug. The above impurity limits are expressed as percentages of the label drug content in the composition.

In another aspect, betamethasone dipropionate compositions of the present application may comprise one or more unknown impurities. One of such an impurity of the betamethasone dipropionate is enol aldehyde impurity (Impurity D). Enol aldehydes are known to be degradation products of corticosteroids having 1, 3-dihydroxyacetone side chain on their D-ring, such as betamethasone, dexamethasone, beclomethasone and the like. Enol aldehyde impurities formed from these corticosteroids via acid-catalysed beta elimination of water from side chain and enol aldehydes could also be formed from the corresponding 17, 21-diesters of these corticosteroids in alkaline conditions.

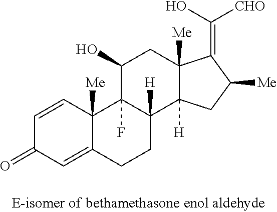

Various conditions such as pH of the composition, and storing conditions influences the formation of enol aldehyde and the enol aldehyde is known to exist in two different isomers E-isomer and Z-isomer. However, the ratio between E and Z isomers may be different depending on the conditions such as pH of the formulation, medium, and temperature. It has been found that E-isomer formation is increased by increase in temperature.

In another aspect, betamethasone propionate compositions of the present application may comprise Impurity D in the amounts of from about 0.001% to about 1.3% of the label drug content.

Surprisingly, in one aspect of the application, the enol aldehyde impurity is controlled well below 1% for period of at least 6 months at 25° C., or for a period of at least 12 months at 25° C., or for a period of at least 18 months at 25° C., or for a period of at least 24 months at 25° C.

In an aspect, the topical spray composition of the present application is substantially free of enol aldehyde impurity for a period of at least 12 months when stored at 2-8° C.

In another aspect, the above topical spray composition comprises betamethasone Dipropionate and oleyl alcohol.

The following examples are provided to illustrate certain specific aspects and embodiments of the application, and are not to be construed as limiting the scope of the application in any manner. The following examples may include compilations of data that are representative of data gathered at various times during the course of development and experimentation related to the present invention.

### EXAMPLES

### Example 1: Exemplary Compositions and Manufacturing Same

In the examples, the active agent betamethasone dipropionate used had a particle size distribution wherein half of the particles had sizes less than about 50 μm, and 90% of the particles had sizes less than about 300 μm.

Exemplary Betamethasone Spray Compositions:

| | Examples | | | | | |
|---|---|---|---|---|---|---|
| | Composition 1 | Composition 2 | Composition 3 | Composition 4 | Composition 5 | Composition 6 |
| Ingredients | w/w | w/w | w/w | w/w | w/w | w/w |
| Betamethasone | 0.0643 | 0.0643 | 0.0643 | 0.0643 | 0.0643 | 0.0643 |
| Dipropionate | | | | | | |
| Sorbitan monostearate | 4.58 | 4.58 | 4.58 | 4.58 | 4.58 | 4.58 |
| Polyoxyl 20 | 2.42 | 2.42 | 2.42 | 2.42 | 2.42 | 2.42 |
| Cetostearyl Ether | | | | | | |
| Cetostearyl alcohol | 1 | 1 | 1 | 1 | 1 | 1 |
| Mineral Oil | 7.06 | 7.06 | 7.06 | 7.06 | 7.06 | 7.06 |
| Oleyl Alcohol | — | — | — | — | — | 5 |
| Elaidyl alcohol | 5 | — | — | — | — | — |
| Caproic alcohol | — | 5 | — | — | — | — |
| Lauryl alcohol | — | — | 5 | — | — | — |
| Stearyl alcohol | — | — | — | 5 | — | — |
| Behenyl alcohol | — | — | — | — | 5 | — |
| Propyl paraben | 0.8 | 0.8 | 0.8 | 0.8 | 0.8 | 0.8 |
| Methyl paraben | 0.2 | 0.2 | 0.2 | 0.2 | 0.2 | 0.2 |
| Butylated hydroxy | 0.05 | 0.05 | 0.05 | 0.05 | 0.05 | 0.05 |
| toluene | | | | | | |
| Hydroxyethyl | 0.05 | 0.05 | 0.05 | 0.05 | 0.05 | 0.05 |
| cellulose | | | | | | |
| Purified water | 78.7757 | 78.7757 | 78.7757 | 78.7757 | 78.7757 | 78.7757 |

Manufacturing Process:
i. Drug Solution Preparation: Betamethasone dipropionate and butylated hydroxyl toluene were solubilized in oleyl alcohol with constant stirring;
ii. Oil Phase preparation: Sorbitan monostearate, polyoxyl 20 cetostearyl ether, cetostearyl alcohol and mineral oil were heated in a stainless steel container up to 70±2° C. Propyl paraben was added to the oil phase;
iii. Drug solution prepared in step 1 was slowly added to oil phase under stirring. Temperature of the stainless steel vessel under 70±2° C.;
iv. Aqueous phase preparation: water and methyl paraben was homogenized and added a quantity of hydroxyl ethyl cellulose to prepare aqueous phase;
v. Oil phase and aqueous phase were homogenized. Homogenization was continued for 10 minutes at 2400 rpm and
vi. Then, the vessel was cooled at 30° C.±2° C. under stirring at 250 rpm using water jacket and allowed to cool to ambient temperature.

Additional Exemplary Compositions:

| Exemplary | Details of solution compositions of betamethasone |
|---|---|
| Compositions | dipropionate |
| Composition 7 | Solution of betamethasone dipropionate |
| | (0.0643% w/w) in Ethanol + Propylene Glycol |
| | (1:1) + Eladiyl alcohol (5% w/w) |
| Composition 8 | Solution of betamethasone dipropionate |
| | (0.0643% w/w) in Ethanol + Propylene Glycol (1:1) + |
| | Caproic alcohol (5% w/w) |
| Composition 9 | Solution of betamethasone dipropionate |
| | (0.0643% w/w) in Ethanol + Propylene Glycol |
| | (1:1) + Lauryl alcohol (5% w/w) |
| Composition | Solution of betamethasone dipropionate |
| 10 | (0.0643% w/w) in Ethanol + Propylene Glycol (1:1) + |
| | Stearyl alcohol (5% w/w) |
| Composition | Solution of betamethasone dipropionate |
| 11 | (0.0643% w/w) in Ethanol + Propylene Glycol (1:1) + |
| | Behenyl alcohol (5% w/w) |
| Composition | Solution of betamethasone dipropionate |
| 12 | (0.0643% w/w) in Ethanol + Propylene Glycol |
| | (1:1) + Oleyl alcohol (5% w/w) |
| Composition | Solution of betamethasone dipropionate |
| 13 | (0.0643% w/w) in Ethanol + Propylene Glycol |
| | (1:1) + Menthol (5% w/w) |
| Composition | Solution of betamethasone dipropionate |
| 14 | (0.0643% w/w) in Ethanol + Propylene Glycol |
| | (1:1) + Alpha Terpeniol (5% w/w) |
| Composition | Solution of betamethasone dipropionate |
| 15 | (0.0643% w/w) in Ethanol + Propylene Glycol |
| | (1:1) + Transcutol (5% w/w) |
| Composition | Solution of betamethasone dipropionate |
| 16 | (0.0643% w/w) in Ethanol + Propylene Glycol |
| | (1:1) + Isopropyl Myristate (5% w/w) |

### Example 2: Stability Testing of Exemplary Composition 6

The prepared formulations, filled into closed containers, were exposed to the stability testing conditions: 25° C. and 60% relative humidity (RH), 30° C. and 65% RH, and 40° C. and 75% RH for two months. All samples remained off-white homogenous emulsions with no phase separation. Drug assay values are within the specified limits of 90-110% of the label drug Content.

The results of studies at various storage points are shown in Table 1, where the values are percentages of the label drug content.

TABLE 1
Results of Stability Studies

| | | Drug | Impurities | | | | |
|---|---|---|---|---|---|---|---|
| Storage Conditions | | Assay | A | B | C | D | Total |
| Initial | — | 100.2 | 0.14 | 0.03 | ND | ND | 0.17 |
| 15 days | 2-8° C. | 100.1 | ND | ND | ND | ND | 0.00 |
| | 25° C. | 101.4 | ND | 0.02 | ND | ND | 0.02 |
| | 30° C. | 99.6 | 0.15 | 0.06 | ND | ND | 0.21 |
| | 40° C. | 100.1 | 0.07 | 0.03 | ND | ND | 0.10 |
| 1 Months | 2-8° C. | 101.3 | 0.03 | ND | ND | ND | 0.03 |
| | 25° C. | 101.2 | 0.06 | 0.05 | ND | ND | 0.11 |
| | 30° C. | 100.9 | 0.09 | 0.05 | ND | ND | 0.14 |
| | 40° C. | 100.4 | 0.20 | 0.15 | ND | 0.02 | 0.59 |
| 2 Months | 2-8° C. | 102.8 | 0.04 | 0.05 | ND | ND | 0.09 |
| | 25° C. | 103.5 | 0.10 | 0.07 | ND | ND | 0.17 |
| | 30° C. | 103.3 | 0.11 | 0.08 | ND | ND | 0.18 |
| | 40° C. | 102.2 | 0.32 | 0.38 | ND | 0.44 | 1.13 |
| 3 Months | 2-8° C. | 99.5 | ND | 0.08 | ND | ND | 0.08 |
| | 25° C. | 100.0 | 0.13 | 0.06 | ND | 0.07 | 0.27 |
| | 30° C. | 100.1 | 0.20 | 0.13 | ND | 0.15 | 0.49 |
| | 40° C. | 98.0 | 0.38 | 0.56 | ND | 0.68 | 1.62 |
| 6 Months | 2-8° C. | 101.2 | 0.09 | ND | ND | ND | 0.09 |
| | 25° C. | 102.2 | 0.21 | 0.15 | ND | 0.15 | 0.51 |
| | 30° C. | 100.0 | 0.35 | 0.31 | ND | 0.3 | 0.97 |
| | 40° C. | 98.8 | 0.67 | 1.09 | ND | 1.12 | 2.98 |
| 9 Months | 2-8° C. | 100.7 | 0.11 | ND | ND | ND | 0.11 |
| | 25° C. | 102.5 | 0.23 | 0.20 | ND | 0.17 | 0.60 |
| | 30° C. | 100.9 | 0.39 | 0.47 | ND | 0.44 | 1.30 |
| 12 Months | 2-8° C. | 102.5 | 0.16 | 0.12 | ND | ND | 0.28 |
| | 25° C. | 96.2 | 0.30 | 0.26 | ND | 0.12 | 0.68 |
| | 30° C. | 98.4 | 0.46 | 0.55 | ND | 0.37 | 1.38 |
| 18 Months | 2-8° C. | 101.2 | 0.18 | 0.15 | ND | 0.14 | 0.47 |
| | 25° C. | 103.1 | 0.36 | 0.41 | ND | 0.31 | 1.08 |
| 24 Months | 2-8° C. | 100.2 | 0.27 | 0.20 | ND | 0.13 | 0.60 |
| | 25° C. | 101.7 | 0.43 | 0.51 | ND | 0.39 | 1.33 |

ND = not detected

### Example 3: Topical Absorption and Penetration Testing of Exemplary Compositions 1-16

Topical spray compositions (Compositions 1-16) were screened for the penetration of drug into different layers of skin and permeation into the receptor phase by finite dosing method using vertical diffusion cells (Franz-type)

Methods and Materials: There were sixteen treatment groups (n=9 cells for each). Each group was having 3 skin samples received from 3 different donors (55 years old or younger; 3 donors×3 replicates). All the test compositions were stored at room temperature.

Skin model: Human cadaver skin was used in this study. The dermatomed human cadaver skin tissue with average thickness of about 350-450 μm. The donor tissue was divided evenly among the diffusion cells.

In vitro percutaneous absorption and penetration study: the topical spray compositions of Compositions 1-16 were screened using vertical diffusion cells (Franz-type). The skin samples were mounted on individual diffusion cells. Each cell in the station was having diffusion area of 0.503 cm² (8 mm in diameter). Each individual cell was static Franz-cell type. The receptor chamber was filled with 3.0 ml of 4% BSA in water supplemented with 0.01% gentamicin sulfate, which will be vigorously and continually mixed. The temperature was set at 32±0.2° C. The cell was incubated at 32° C. for one hour before dosing.

At end of incubation period, samples from the receptor fluid were taken as the t=0 samples. A fresh batch of receptor fluid pre-incubated at 32° C. was introduced into the receptor chamber in the HTS cells. The compositions were dosed at a level of approximately 2.5 mg per cell, which was equivalent to 5 mg/cm² and they were applied using a positive displacement pipette. The dosing volumes were calculated by calculating density of each composition. The samples were collected at the time intervals of 0 hour, 2 hours, 6 hours, 10 hours, 12 hours or 24 hours and stored in a freezer before analysis. Skin penetration was analyzed by samples collected at 0, 2, 6, 10, 12 or 24 hours and the full mass balance study was conducted after 24 hours.

The full mass balance study amount of betamethasone dipropionate and its metabolites were analyzed from following skin locations: skin surface (unabsorbed, and/or unpenetrated), stratum corneum (from tape stripping), and epidermis (separation from dermis and followed by solvent extraction), dermis (separation from epidermis and followed by solvent extraction). The tissue surface was wiped with Q-tip wetted with 1×PBS three times to remove unabsorbed and unpenetrated API (i.e.,) betamethasone dipropionate and its metabolites. The standard tape-stripping method was used to remove the stratum corneum (SC) layer. After removal of stratum corneum layer, the remaining tissue was wetted with 1×PBS, epidermis and dermis layers were separated mechanically.

All collected samples were analyzed using LC-MS/MS with qualified method for following analytes: betamethasone dipropionate, betamethasone-17-propionate, betamethasone-21-propionate and betamethasone base.

TABLE 2
Betamethasones retained in skin layers

| | Stratum | | | |
|---|---|---|---|---|
| Exemplary | Corneum | Epidermis | Dermis | |
| Compositions | (ng) | (ng) | (ng) | Total (ng) |
| Composition 1 | 5.40 | 17.09 | 14.22 | 36.71 |
| Composition 2 | 11.11 | 15.23 | 4.87 | 31.21 |
| Composition 3 | 36.83 | 42.55 | 33.81 | 113.19 |
| Composition 4 | 2.02 | 1.89 | 0.79 | 4.70 |
| Composition 5 | 17.57 | 7.43 | 6.38 | 31.39 |
| Composition 6 | 33.00 | 18.34 | 14.11 | 65.45 |
| Composition 7 | 82.65 | 198.11 | 40.52 | 321.28 |
| Composition 8 | 78.76 | 244.14 | 57.68 | 380.59 |
| Composition 9 | 78.61 | 162.92 | 67.49 | 309.01 |
| Composition | 70.48 | 199.21 | 49.78 | 319.47 |
| 10 | | | | |
| Composition | 76.09 | 182.91 | 86.33 | 345.32 |
| 11 | | | | |
| Composition | 73.29 | 206.15 | 54.89 | 334.32 |
| 12 | | | | |
| Composition | 108.23 | 197.96 | 53.02 | 359.21 |
| 13 | | | | |
| Composition | 141.82 | 165.20 | 68.15 | 375.17 |
| 14 | | | | |
| Composition | 184.39 | 133.52 | 106.10 | 424.01 |
| 15 | | | | |
| Composition | 236.14 | 98.17 | 87.07 | 421.39 |
| 16 | | | | |

TABLE 3
Percentage of betamethasones retained in skin
layers and receptor level

| | | | Receptor | Percentage | |
|---|---|---|---|---|---|
| | | Skin | level | of | Percentage |
| | Applied | retention | after 24 | retention | permeated |
| Exemplary | dose | dose (in | Hours | in skin | into |
| Compositions | (in ng) | ng) | (in ng) | layers | receptor |
| Composition 1 | 1500 | 36.71 | 11.26 | 2.45 | 0.75 |
| Composition 2 | 1360 | 31.21 | 19.35 | 2.29 | 1.42 |
| Composition 3 | 1520 | 113.19 | 50.69 | 7.45 | 3.33 |
| Composition 4 | 745 | 4.70 | 2.31 | 0.63 | 0.31 |
| Composition 5 | 1370 | 31.39 | 2.78 | 2.29 | 0.20 |
| Composition 6 | 1255 | 65.45 | 11.82 | 5.22 | 0.94 |
| Composition 7 | 1340 | 321.28 | 188.78 | 23.98 | 14.09 |
| Composition 8 | 1385 | 380.59 | 124.92 | 27.48 | 9.02 |
| Composition 9 | 1340 | 309.01 | 130.58 | 23.06 | 9.74 |
| Composition | 1520 | 319.47 | 132.89 | 21.02 | 8.74 |
| 10 | | | | | |
| Composition | 1445 | 345.32 | 241.54 | 23.90 | 16.72 |
| 11 | | | | | |
| Composition | 1190 | 334.32 | 16.64 | 28.09 | 1.40 |
| 12 | | | | | |
| Composition | 1165 | 359.21 | 50.69 | 30.83 | 4.35 |
| 13 | | | | | |
| Composition | 1160 | 375.17 | 183.24 | 32.34 | 15.80 |
| 14 | | | | | |
| Composition | 1055 | 424.01 | 237.29 | 40.19 | 22.49 |
| 15 | | | | | |
| Composition | 1300 | 421.39 | 211.86 | 32.41 | 16.30 |
| 16 | | | | | |

TABLE 4
Skin retention ratio range (n = 9 cells)

| Exemplary | | |
|---|---|---|
| Compositions | Minimum | Maximum |
| Composition 1 | 1.4 | 8.4 |
| Composition 2 | 0.7 | 8.3 |
| Composition 3 | 1.1 | 13.7 |
| Composition 4 | 0.9 | 7.3 |
| Composition 5 | 0.7 | 49.6 |
| Composition 6 | 1.5 | 47.7 |
| Composition 7 | 0.2 | 10.5 |
| Composition 8 | 1.3 | 10.4 |
| Composition 9 | 0.9 | 10.0 |
| Composition 10 | 0.6 | 24.1 |
| Composition 11 | 0.4 | 3.9 |
| Composition 12 | 5.7 | 82.1 |
| Composition 13 | 2.8 | 81.5 |
| Composition 14 | 0.7 | 3.8 |
| Composition 15 | 0.3 | 10.1 |
| Composition 16 | 1.1 | 5.0 |

### Example 4: Irritation Patch Test Study of Betamethasone Dipropionate Spray

Total forty (40) subjects were enrolled and out of which thirty four (34) had completed the study. This was a randomized, double-blind, single-center, vehicle-controlled, within-subject comparison patch test study of followings for irritation potential when repeatedly applied to the skin under semi-occlusive conditions in healthy volunteers:
i. Betamethasone dipropionate Spray (Exemplary Composition 6),
ii. Vehicle spray (without active agent of Composition 6),
iii. Vehicle lotion (isopropyl alcohol, hydroxypropyl cellulose, sodium phosphate monobasic monohydrate, propylene glycol, phosphoric acid, sodium hydroxide and water i.e. vehicle for Diprolene lotion 0.05%,
iv. Sodium lauryl sulfate (SLS) 0.2% and
v. Saline 0.9%

All subjects received applications of each study product to intact skin at randomly assigned, adjacent sites on the back. Evaluators and subjects were blinded and unaware of the identity of the study product at the patch test sites. The study products were applied under semi-occlusive patch conditions using a 2 cm×2 cm patch. The products were applied to either side of the infrascapular area of the back. Evaluation of dermal reactions at the application sites were assessed clinically using a visual scale that rates the degree of erythema, edema, and other signs of cutaneous irritation.

A total of 21 patch applications were made over a period of 21 days. Irritancy score of each composition was recorded.

### Conclusion

Significantly more irritation was observed at the Vehicle Lotion site and 0.2% SLS site than at the betamethasone dipropionate spray site, vehicle spray site and 0.9% sterile saline site. There was no significant difference in irritation between the vehicle lotion site and 0.2% SLS site, and no significant difference in irritation between the betamethasone dipropionate spray site and vehicle spray site, the betamethasone dipropionate spray site and 0.9% sterile saline site. Under the exaggerated conditions of use in this study, with continuous exposure under semi occlusion for 21 days, betamethasone dipropionate spray (Composition 6) and its vehicle spray (Composition 6 without active agent) produced no evidence of significant irritation. In comparison, the vehicle lotion (vehicle for DIPROLINE Lotion augmented 0.05%) produced mild irritation, as there was no significant difference in irritation between the Vehicle Lotion site and 0.2% SLS site, a known mild irritant.

### Example 5: Spray Characteristics of Exemplary Composition 6

The spray pattern characterizes the spray following impaction on an appropriate target, i.e., a thin layer chromatography (TLC) plate. A TLC plate having silica gel 60, F254 (florescence indicator), 250 μm thick layer on glass was used as target in present study and the TLC plate was held with suitable fastener.

Automatic air pressure actuation device were used in the study to automate the spray actuations. Mark VII® Max pumps (1-10) were used to pump the composition in the spray pattern studies. The spray distance was 40 mm from the spray nozzle to the TLC plate. The sprayer (the container is a 2 oz HDPE bottle) was loaded with exemplary compositions and the composition density was 0.9081 g/ml and Kern ALJ220-4NM was used to measure the output from each stroke. Compositions were shaken three times before priming and priming the pump 10× into a hood was done to ensure a full stroke. Sprayer and TLC plate with fastener were brought into right position at 40 mm distance. Actuation profile was chosen as the pump output is 0.16 ml; actuation/return Velocity was 100 mm/s; actuation/return acceleration was 5700 mm/s2; initial delay was 0 ms; hold time was 100 ms; final delay was 0 ms and inter actuation delay was 0 ms. After spray, the TLC plate was taken away from the fastener and the spray pattern was viewed under 254 nm UV light and a suitable camera was used to take pictures (e.g., Digital camera) in ultraviolet light and minimum and maximum diameters of spray patterns were determined. This test was repeated for 28 days (2 times a day) and compositions were stored in room temperature horizontally and upright positions between the daily tests.

### Example 6: Fractional Solubility Study of Exemplary Composition 6 for Assessing Distribution of Betamethasone Dipropionate in Oil/Aqueous Phase

Fractional solubility study was performed to assess betamethasone dipropionate distribution in oil-in-water emulsion composition. Exemplary Composition 6 was accurately weighed about 3.1 g and transferred into in 15 ml centrifuge tube. About 3.1 g of sodium chloride was added in the same centrifuge tube containing the composition and was shaken well. 10 ml of water from TKA water purification system was added to the centrifuge tube and was shaken well for 2 minutes for emulsion breaking. The centrifuge tube was loaded in the centrifuge and centrifuged the composition at 10000 rpm at 15° C. for 5 minutes. After centrifugation, two distinct layers were observed. It was concluded based on the volume that the upper layer was oil phase (White cream in description) and the lower layer was aqueous layer (Clear colorless liquid in description).

The lower layer was injected in the HPLC and analyzed using the HPLC standard test procedure for assay and no betamethasone dipropionate peak was observed. The upper layer was carefully transferred into 100 ml volumetric flask and drug content assay was performed as per the HPLC standard test procedure for assay. It was observed that the assay of betamethasone dipropionate in the upper layer (i.e., oil phase) was about 100.3%.

### Example 7: Hypothalamic-Pituitary-Adrenal (HPA) Axis Suppression Study

The potential of spray Composition 6 to suppress the HPA axis was evaluated in subjects with moderate to severe plaque psoriasis under maximal use conditions. Seventy-five subjects were randomized to treatment with either Composition 6 with 15-day treatment (n=23), Composition 6 with 29-day treatment (n=25), or Diprolene Lotion, 0.05% with 15-day treatment (n=27). The treatments were applied twice daily. Subjects had 20% to 50% body surface area treated to achieve maximal use exposure. The target dose was at least 5 to 7 g per day. HPA axis function was tested using an ACTH stimulation test with ≥18 μg/dL designated as the normal post-stimulation cortisol level.

Plasma cortisol was determined before and after adrenocorticotropic hormone (ACTH) stimulation in subjects with moderate to severe psoriasis treated twice daily with Composition (15 days and 29 days) or Diprolene lotion 0.05% (15 days). The incidence of Treatment-Emergent Adverse Event (TEAEs) was similar across all treatment groups (25.9% to 32%). No serious TEAEs or discontinuations from the study due to TEAE were reported in any treatment group. Application site pruritus was reported in all treatment groups at a similar incidence: (4%, 7.4% and 4.5% for Composition 6 in 15 days treatment group, Composition 6 in 29 days treatment group and Diprolene lotion, 0.05% in 15 days treatment group respectively). An additional TEAE of application site burning/stinging (coded to application site pain), was reported only for one subject in the Composition 6 in 29 days treatment group (3.7%).

Among 68 evaluable subjects, abnormal ACTH stimulation test results suggestive of adrenal suppression were identified in 5 out of 24 (20.8%) subjects after treatment with Composition 6 twice daily for 15 days and in 5 out of 20 (25.0%) subjects after treatment with Diprolene Lotion, 0.05% twice daily for 15 days. No subject (0 out of 24) had abnormal ACTH stimulation test results after treatment with Composition 6 twice daily for 29 days.

The incidence of HPA axis suppression was similar between the Composition 6 in 15 days treatment group and Diprolene lotion, 0.05% in 15 day treatment group at 20.8% and 25.0%, respectively (Table 5). HPA axis suppression was not observed for Composition 6 in 29 days treatment group.

TABLE 5
Summary of ACTH Stimulation Test at End of
Treatment (Safety Population-Subjects with Data)

| ACTH | Composition 6 | Composition 6 | DIPROLENE lotion |
|---|---|---|---|
| Stimulation | 15 days (Number | 29 days | 0.05% 15 days |
| Test | of subjects = | (number of | (Number of |
| Results | 24) | subjects = 24) | subjects = 20) |
| Normal | 19 (79.2%) | 24 (100.0%) | 15 (75.0%) |
| Abnormal | 5 (20.8%) | 0 (0.0%) | 5 (25.0%) |

Betamethasone dipropionate metabolites i.e. Betamethasone-17-propionate and betamethasone base were detected in the majority of the subjects. Subjects in the Composition 6 of the 29 days treatment group had lower ‘median Cmax’ for betamethasone-17-propionate and betamethasone base plasma concentrations when compared with that of Composition 6 in 15 days treatment group (Table-6)

TABLE 6
Median maximum plasma concentrations (‘median
Cmax’ in pg/mL) of betamethasone-17-propionate and betamethasone
base after treatment 15 and 29 days groups with Composition 6.

| | Composition 6 b.i.d | Composition 6 b.i.d |
|---|---|---|
| Analyte | 15 days | 29 days |
| Betamethasone- | 65 (9, 490) | 52 (10, 225) |
| 17-propionate | | |
| Betamethasone | 54 (5, 761) | 42 (10, 223) |
| base | | |

Data represents median of maximum plasma concentrations (‘median Cmax’) (minimum, maximum)

TABLE 7
Mean maximum plasma concentrations (‘mean Cmax’
in pg/ml) of betamethasone-17-propionate and betamethasone base
after treatment 15 and 29 days groups with Composition 6:

| | Example 6 composition | Example 6 composition |
|---|---|---|
| | b.i.d | b.i.d |
| Analyte | 15 days | 29 days |
| Betamethasone- | 120 (9, 490) | 64 (10, 225) |
| 17-propionate | | |
| Betamethasone | 119 (5, 761) | 58 (10, 223) |
| base | | |

Data represents mean of maximum plasma concentrations (‘mean Cmax’) (minimum, maximum)

What is claimed is:

1. An aqueous based topical spray composition comprising:
a) a betamethasone compound;
b) a fatty alcohol selected from oleyl alcohol, elaidyl alcohol, caproic alcohol, linoleyl alcohol, linolenyl alcohol, lauryl alcohol, stearyl alcohol, behenyl alcohol, cetostearyl alcohol, 2-heptyl-1-undecanol, 1,17-hepatadecanediol, and combinations thereof;
c) at least one emulsifying agent; and
d) water;
wherein said composition is stable at least for the period of about 6 months at 40° C. or at least for the period of 24 months at 25° C.

2. The composition of claim 1, wherein said composition does not form a film layer at the application site.

3. The composition of claim 1, wherein said fatty alcohol is oleyl alcohol.

4. The composition of claim 3, wherein said composition further comprises one or more other fatty alcohols.

5. The composition of claim 4, wherein said one or more other fatty alcohols are selected from the group consisting of elaidyl alcohol, linoleyl alcohol, linolenyl alcohol, caproic alcohol, lauryl alcohol, stearyl alcohol, behenyl alcohol, cetostearyl alcohol, 2-heptyl-1-undecanol, 1,17-hepatadecanediol, and combinations thereof.

6. The composition of claim 3, wherein said betamethasone compound is present in amounts equivalent to about 0.025 percent to about 0.1 percent by weight of the total composition.

7. The composition of claim 6, wherein said betamethasone compound is betamethasone dipropionate.

8. The composition of claim 7, wherein said oleyl alcohol is present in an amount of about 0.001 percent to about 15 percent by weight of total composition.

9. The composition of claim 8, wherein said composition further comprises cetostearyl alcohol.

10. The composition of claim 3, wherein said emulsifying agent is selected from the group consisting of a cationic surfactant, an anionic surfactant, a zwitterionic surfactant, an amphoteric surfactant, and combinations thereof.

11. The composition of claim 3, wherein said emulsifying agent is a nonionic surfactant.

12. The composition of claim 3, wherein the composition further comprises a pharmaceutically and/or dermatologically acceptable excipient.

13. The composition of claim 12, wherein said excipient is a polymer selected from the group comprising of a carbomer, polyethylene glycol, acrylate polymer, methacrylate polymer, polyvinylpyrrolidone, copolymer based on butyl methacrylate and methyl methacrylate, vinyl acetate, polyvinyl acetate, cellulose, gum, alginate, cellulose acetate phthalate, cellulose acetate butyrate, hydroxypropyl methylcellulose phthalate, and combinations thereof.

14. The composition of claim 3, further comprising one or more antioxidant, preservative, humectant or plasticizer.

15. The composition of claim 3, wherein said composition is substantially non-irritating to the subject's skin.

16. The composition of claim 3, wherein said betamethasone compound is selected from the group comprising of betamethasone benzoate, betamethasone dipropionate, betamethasone sodium phosphate, betamethasone valerate, and combinations thereof.

17. The composition of claim 3, wherein said composition is free of propylene glycol, substantially non-foaming, substantially free of propellant, and substantially non-occlusive to the skin.

18. The composition of claim 3, wherein said composition is substantially free of enol aldehyde impurity at least for the period of about 12 months when stored at 2-8° C.

19. The composition of claim 3, wherein said composition does not form any film layer at the application site.

20. An aqueous based topical spray composition comprising:
a) a betamethasone compound;
b) an oil phase comprising:
i. a fatty alcohol selected from oleyl alcohol, elaidyl alcohol, caproic alcohol, linoleyl alcohol, linolenyl alcohol, lauryl alcohol, stearyl alcohol, behenyl alcohol, cetostearyl alcohol, 2-heptyl-1-undecanol, 1,17-hepatadecanediol, and combinations thereof; and
ii. an emulsifying agent; and
c) an aqueous phase comprising water;
wherein a weight ratio between said oil phase and said aqueous phase is from about 1:1.5 to about 1:4; and
wherein said composition is free of propylene glycol, substantially non-foaming, substantially free of propellant, and substantially non-occlusive to the skin.

21. The composition of claim 20, wherein said fatty alcohol is oleyl alcohol.

22. The composition of claim 21, wherein the composition is stable at least for the period of about 6 months at 40° C. or at least for the period of 24 months at 25° C.

23. The composition of claim 21, wherein the weight ratio between said oil phase and said aqueous phase is selected from about 1:1.5 or about 1:1.6 or about 1:1.7 or about 1:1.8 or about 1:1.9 or about 1:2 or about 1:2.1 or about 1:2.2 or about 1:2.3 or about 1:2.4 or about 1:2.5 or about 1:2.6 or about 1:2.7 or about 1:2.8 or about 1:2.9 or about 1:3 or about 1:3.1 or about 1:3.2 or about 1:3.3 or about 1:3.4 or about 1:3.5 or about 1:3.6 or about 1:3.7 or about 1:3.8 or about 1:3.9 or about 1:4.

24. The composition of claim 21, wherein a D90 of the dispersed phase droplets is in the range of from about 1 μm to about 10 μm.

25. The composition of claim 21, wherein said composition is substantially free of enol aldehyde impurity at least for the period of about 12 months when stored at 2-8° C.

26. The composition of claim 21, wherein said composition does not form a film layer at the application site.

27. The composition of claim 21, wherein said emulsifying agent is selected from the group consisting of a cationic surfactant, an anionic surfactant, a zwitterionic surfactant, an amphoteric surfactant, nonionic surfactant, and combinations thereof.

28. The composition of claim 21, wherein said emulsifying agent is a nonionic surfactant.

29. The composition of claim 21, wherein said betamethasone compound is selected from the group comprising of betamethasone benzoate, betamethasone dipropionate, betamethasone sodium phosphate, betamethasone valerate, and combinations thereof.

30. The composition of claim 21, wherein said betamethasone compound is betamethasone dipropionate.
